(19) United States
(12) Patent Application Publication
(10) Pub. No.: US 2005/0090789 A1
(43) Pub. Date: Apr. 28, 2005

(54) Absorbent composite having improved surface dryness

(76) Inventors: Peter A. Graef, Puyallup, WA (US); Terry M. Grant, Auburn, WA (US); David G. Marsh, Federal Way, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 10/994,023

(22) Filed: Nov. 19, 2004

Related U.S. Application Data
(63) Continuation of application No. 09/815,933, filed on Mar. 23, 2001, now abandoned.
Application No. 09/815,933 is a continuation-in-part of application No. 09/326,213, filed on Jun. 4, 1999, now Pat. No. 6,734,335.
Application No. 09/326,213 is a continuation-in-part of application No. 09/137,503, filed on Aug. 20, 1998, now abandoned.
Application No. 09/137,503 is a continuation of application No. PCT/US97/22342, filed on Dec. 5, 1997.
Application No. 09/815,933 is a continuation-in-part of application No. 09/569,380, filed on May 11, 2000, now abandoned.
Application No. 09/569,380 is a continuation-in-part of application No. PCT/US99/26560, filed on Nov. 10, 1999.
Application No. 09/815,933 is a continuation-in-part of application No. 09/141,152, filed on Aug. 27, 1998.
(60) Provisional application No. 60/032,916, filed on Dec. 6, 1996.
(60) Provisional application No. 60/107,998, filed on Nov. 11, 1998.
(60) Provisional application No. 60/046,395, filed on May 13, 1997.

(51) Int. Cl. A61F 13/15; A61F 13/20

(52) U.S. Cl. 604/368

(57) ABSTRACT

The present invention provides an absorbent composite having improved surface dryness. The composite has three strata with adjacent strata separated by a transition zone. The composite's first stratum includes synthetic fibers and provides the composite with improved surface dryness. Methods for forming the composite and absorbent articles that incorporated the composite are also provided.

### CROSS-REFERENCES TO RELATED APPLICATIONS

This application is a continuation-in-part of copending U.S. patent application Ser. No. 09/326,213, filed Jun. 4, 1999, which is a continuation-in-part of U.S. patent application Ser. No. 09/137,503, filed Aug. 20, 1998, which is a continuation of international application number PCT/US97/22342, filed Dec. 5, 1997, which is a continuation-in-part of U.S. patent application Ser. No. 60/032,916, filed Dec. 6, 1996; a continuation-in-part of copending U.S. patent application Ser. No. 09/569,380, filed May 11, 2000; which is a continuation-in-part of international application number PCT/US99/26560, filed Nov. 10, 1999; which is a continuation-in-part of U.S. patent application Ser. No. 60/107,998, filed Nov. 11, 1998; and a continuation-in-part of copending U.S. patent application Ser. No. 09/141,152, filed Aug. 27, 1998, which is a continuation of international application number PCT/US98/09682, filed May 12, 1998, which is a continuation-in-part of U.S. patent application Ser. No. 60/046,395, filed May 13, 1997; priority of the filing dates of which is hereby claimed under 35 U.S.C. § § 120 and 119, respectively. This application claims the benefit of the priority of U.S. patent application Ser. No. 60/191,870, filed Mar. 23, 2000. Each of these applications is incorporated herein by reference in its entirety.

### FIELD OF THE INVENTION

The present invention relates generally to absorbent composites and, in particular, to an absorbent composite having improved surface dryness.

### BACKGROUND OF THE INVENTION

Currently, diapers are manufactured using individual materials and layers that are designed for a specific functionality. In addition to a liquid pervious topsheet and a liquid impervious backsheet, a typical diaper includes a multilayered absorbent structure. The absorbent structure has an acquisition layer for rapidly acquiring a liquid insult, optionally a distribution layer for receiving and distributing liquid acquired from the acquisition layer, and a storage layer for retaining the acquired liquid. These individual layers are assembled on a production line to provide a diaper having a multilayered absorbent core. Not surprisingly, the nature of the interface between these layers affects the product's performance characteristics and functionality. For diapers assembled on a typical diaper production line, there exists a substantial discontinuity between the materials of each layer resulting in a disruption of the liquid communication between these layers, ultimately impeding liquid transfer between these layers. Problems associated with discontinuities between the materials of adjacent layers is ordinarily reduced by using adhesives. However, adhesives tend to hinder liquid transfer.

Accordingly, there exists a need for an absorbent composite for use in an absorbent article, such as a diaper, in which the composite's component layers are in intimate liquid communication such that transfer of liquid between the layers is not hindered. A need also exists for composites having improved surface dryness after liquid acquisition. The present invention seeks to fulfill these needs and provides further related advantages.

### SUMMARY OF THE INVENTION

Currently, diapers are manufactured using individual materials and layers that are designed for a specific functionality. In addition to a liquid pervious topsheet and a liquid impervious backsheet, a typical diaper includes a multilayered absorbent structure. The absorbent structure has an acquisition layer for rapidly acquiring a liquid insult, optionally a distribution layer for receiving and distributing liquid acquired from the acquisition layer, and a storage layer for retaining the acquired liquid. These individual layers are assembled on a production line to provide a diaper having a multilayered absorbent core. Not surprisingly, the nature of the interface between these layers affects the product's performance characteristics and functionality. For diapers assembled on a typical diaper production line, there exists a substantial discontinuity between the materials of each layer resulting in a disruption of the liquid communication between these layers, ultimately impeding liquid transfer between these layers. Problems associated with discontinuities between the materials of adjacent layers is ordinarily reduced by using adhesives. However, adhesives tend to hinder liquid transfer.

Accordingly, there exists a need for an absorbent composite for use in an absorbent article, such as a diaper, in which the composite's component layers are in intimate liquid communication such that transfer of liquid between the layers is not hindered. A need also exists for composites having improved surface dryness after liquid acquisition. The present invention seeks to fulfill these needs and provides further related advantages.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic cross-sectional view of a portion of a representative composite formed in accordance with the present invention;

FIGS. 2A-2C are schematic cross-sectional views of portions of representative composites formed in accordance with the present invention illustrating the composites' transition zones;

FIG. 3 is a diagram of a divided headbox useful for forming a representative composite according to the present invention;

FIG. 4 is a schematic cross-sectional view of a portion of a representative composite formed in accordance with the present invention;

FIG. 5 is a diagrammatic view illustrating a twin-wire device and method for forming the composite of the invention;

FIG. 6 is a diagrammatic view illustrating a headbox assembly and method for forming the composite of the invention;

FIG. 7 is a diagrammatic view illustrating a headbox assembly and method for forming the composite of the invention;

FIG. 8 is a diagrammatic view illustrating conduits for introducing materials into a headbox in accordance with the present invention;

FIG. 9A is a schematic perspective view of a representative construct formed in accordance with the present invention;

FIG. 9B is a schematic cross-sectional view of the construct illustrated in FIG. 9A;

FIG. 10 is a schematic perspective view of a representative C-fold construct formed in accordance with the present invention;

FIGS. 11A-D are schematic views of representative composites formed in accordance with the present invention illustrating softening patterns;

FIG. 12 is a cross-sectional view of a representative absorbent construct incorporating a composite formed in accordance with the present invention;

FIG. 13 is a cross-sectional view of another representative absorbent construct incorporating a composite formed in accordance with the present invention;

FIG. 14 is a cross-sectional view of a further representative absorbent construct incorporating a composite formed in accordance with the present invention;

FIG. 15 is a cross-sectional view of a representative absorbent article incorporating a composite formed in accordance with the present invention;

FIG. 16 is a cross-sectional view of a another representative absorbent article incorporating a composite formed in accordance with the present invention;

FIG. 17 is a cross-sectional view of a further representative absorbent article incorporating a composite formed in accordance with the present invention; and

FIG. 18 is a cross-sectional view of a still another representative absorbent article incorporating a composite formed in accordance with the present invention.

### DETAILED DESCRIPTION OF THE PREFERRED EMBODIMENT

The composite formed in accordance with the present invention is a fibrous composite having three strata. Fibers from adjacent strata are intermixed, commingled, and entangled to provide a nonlaminated stratified composite. The absorbent composites formed in accordance with the present invention are in contrast to conventional multilayered composites that are characterized in having abrupt transitions in material compositions at the interfaces of adjacent layers. The absorbent composites of this invention avoid such abrupt material transitions and are characterized by continuous, nonstepwise material gradients in the transition zones between adjacent strata. The transition zone includes the materials of adjacent strata intermixed and commingled to a substantial degree. The transition zone integrally. and intimately connects adjacent strata of the absorbent composite. The transition zone assures a continuity of material between the zones.

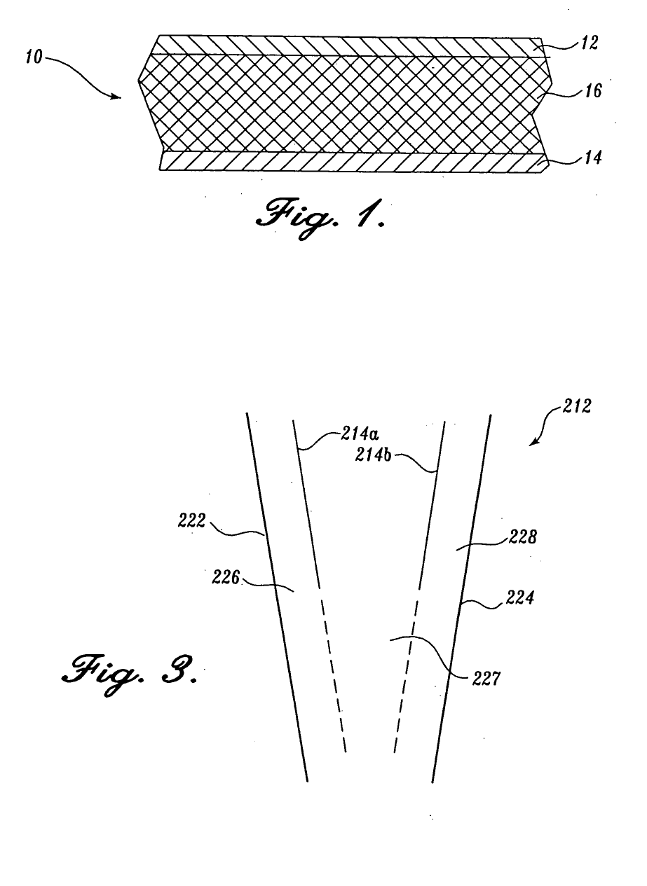

In one aspect, the present invention provides a unitary composite that includes three strata. The term “unitary” refers to the composite's structure in which adjacent strata are integrally connected through a transition zone to provide a structure with adjacent strata in intimate fluid communication. A representative composite is schematically illustrated in FIG. 1. Referring to FIG. 1, composite 10 includes intermediate stratum 16 and coextensive surface strata 12 and 14.

In the composite, transition zones separate the composite's strata. The nature of the transition zone can vary from composite-to-composite and from stratum-to-stratum within a composite. The transition zone can be designed to satisfy the performance requirements of a particular composite. In general, the transition zone integrally connects adjacent strata and provides for intimate liquid communication between strata. The transition zone includes fibers from adjacent strata. A composite having three strata has two transition zones. The first transition zone includes fibers from the first and second strata, and the second transition zone includes fibers from the second and third strata.

The composite's transition zone is located in the composite generally between the substantially homogeneous regions of the individual strata and is defined as the region of the composite where the fibers from one stratum are commingled with fibers from an adjacent stratum.

Transition zone thickness within a composite can be widely varied depending on the composite. Absorbent composites of the present invention can include a transition zone that is relatively thin. Absorbent composites that include such thin transition zones have fairly abrupt transitions in material composition between strata. Alternatively, the composite can include a transition zone that is gradual such that the transition from one zone to the next occurs over a relatively greater thickness of the composite. In such a composite, the material compositions of each zone can be intermixed to a significant extent resulting in rather extended composition gradients.

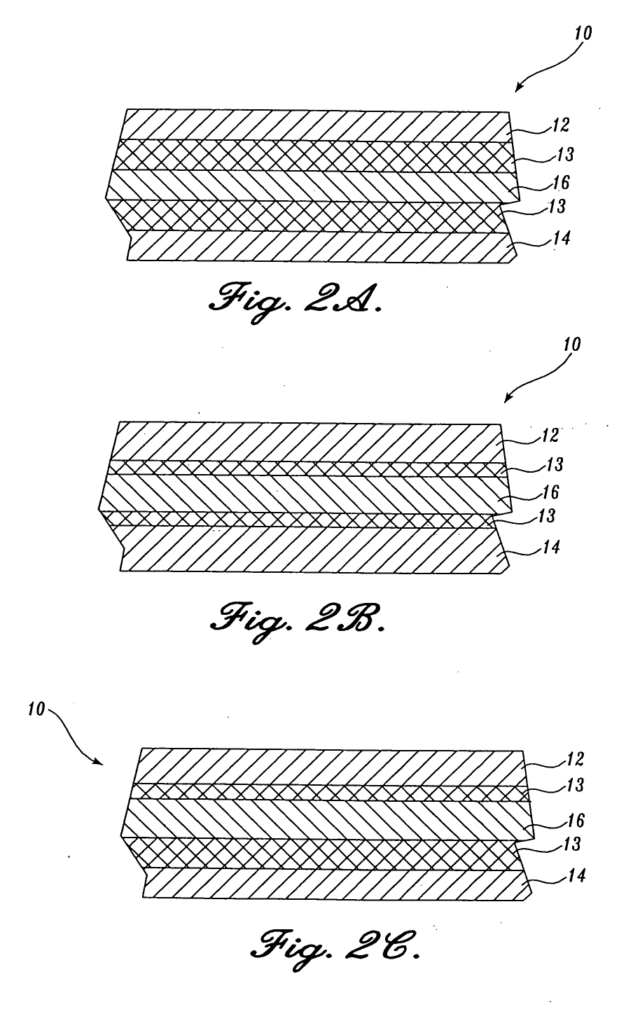

Representative composites formed in accordance with the present invention are schematically illustrated in FIGS. 2A-C. In these figures, the transition zone is illustrated. Referring to FIGS. 2A-C, composite 10 includes intermediate stratum 16 and coextensive surface strata 12 and 14 with adjacent strata separated by transition zone 13.

The composites formed in accordance with the present invention include three strata with adjacent strata separated by a transition zone. In one embodiment, the composites are formed by a method that includes depositing a fibrous furnish on a foraminous support. In the method, the composite's strata can be formed through the use of a divided or multichanneled headbox. For forming composites having three strata, a headbox divided into three chambers can be used. The first stratum can be formed from a first fibrous furnish introduced into a first headbox chamber, the second stratum can be formed from a second fibrous furnish introduced into a second headbox chamber, and the third stratum can be formed from a third fibrous furnish introduced into a third headbox chamber. The deposition of the headbox contents (e.g., from the first, second, and third-chambers) onto a foraminous support provides a web that, on dewatering and drying, provides a representative composite of the invention, a unitary composite having three strata with adjacent strata separated by a transition zone. For the composite described above, the composite's first transition zone results from the mixing of the first and second fibrous furnishes (e.g., in the headbox) and includes materials from both furnishes. Likewise, the composite's second transition zone results from the mixing of the second and third fibrous furnishes (e.g., in the headbox) and includes materials from both furnishes. The composite's transition zone thickness and density can be controlled by the headbox configuration and fiber flow rate. In the divided headbox described above, the first and second furnishes (and the second and third furnishes) mix to an extent prior to exiting the headbox and ejection onto the foraminous support. The greater the mixing prior to ejection from the headbox, the greater the transition zone.

Referring to FIG. 3, headbox 212 includes walls 222 and 224 and dividers (or baffles) 214a and 214b creating first chamber 226, second chamber 227, and third chamber 228. The length of dividers 214a and 214b can be varied such that the point at which furnishes introduced into chambers 226, 227, and 228 meet and commence mixing can be adjusted. The variances in the length of dividers 214a and 214b are depicted as dashed lines in FIG. 3. In accordance with the present invention, the point at which furnishes meet and commence mixing in the headbox (e.g., the length of dividers) need not be the same. By adjusting the point at which furnishes meet, composites having individual strata and transition zones having variable thickness within the composite can be provided. For example, a three-strata composite can have two transitions zones having the same thickness as shown in FIGS. 2A and 2B. Referring to FIGS. 2A and 2B, representative composites 10 have first stratum 12, second stratum 16, third stratum 14, and transition zones 13. The thicker transition zones 13 in FIG. 2A compared to the thinner transition zones 13 in FIG. 2B result from forming using the headbox of FIG. 2 using relatively shorter dividers 214a and 214b. Alternatively, as described above and illustrated in FIG. 2C, representative composite 10 can include transition zones 13 having different thicknesses.

The individual strata of the composites of the invention are formed from fibrous furnishes that include materials specific for performance of the function desired by the particular stratum and the composite as a whole. Accordingly, the composites of the invention can include a variety of materials. In addition to fibrous materials, such as cellulosic and synthetic fibers, the composites (i.e., composites' strata) can include absorbent material, such as superabsorbent polymers, and a binder for increasing the strength of the composite. Other additives commonly incorporated into conventional absorbent composites can also be included.

In one embodiment, the present invention provides a composite that includes a first stratum that includes a hydrophobic fibrous material that does not absorb bodily fluids and which forms an open and bulky stratum having a relatively low basis weight. Preferred components for such a stratum include synthetic fibers including polyester fibers, for example, polyethylene terephthalate (PET) fibers and bicomponent binder fibers. The composite's second stratum (i.e., intermediate stratum) includes a fibrous matrix and absorbent material (e.g., superabsorbent polymer particles). The fibrous matrix can include a mixture of matrix fibers (e.g., fluff pulp fibers) and resilient fibers (e.g., crosslinked cellulosic fibers). Depending on the composite's intended use, the third stratum can have a composition similar to the first stratum as noted above. Alternatively, the third stratum can include a fibrous blend of fluff pulp and crosslinked cellulosic fibers. One or more of the strata can also include a binder to effect bonding between the fibers and other materials of the stratum and/or between the fibers and other materials of adjacent strata.

The composite of the invention can be advantageously incorporated into a variety of absorbent products and articles to provide rapid storage capacity, to increase the liquid acquisition rate, to reduce leakage, and to enhance the rewet and dry feel performance of the absorbent article.

Referring again to FIG. 1, composite 10 includes a first stratum 12, a second stratum 16, and a third stratum 14. The first stratum serves primarily as an acquisition stratum that can rapidly acquire liquid, distribute the liquid throughout the stratum, and then rapidly and efficiently pass the liquid to an underlying stratum. The first stratum can also impart low rewet and dry feel performance to the composite. The first stratum has greater pore size and lower hydrophilicity than the second stratum. The second stratum serves as a liquid storage layer and rapidly withdraws liquid acquired by the first stratum. The third stratum can serve to provide strength to the composite, to impart enhanced liquid distribution to the composite, and to assist in retaining superabsorbent particles within the composite.

In one embodiment, the first stratum is a relatively hydrophobic stratum that includes a hydrophobic fibrous material (i.e., one or more hydrophobic fibers). Other fibers, such as hydrophilic fibers, may be included in the first stratum as long as the first stratum remains relatively less hydrophilic than the second stratum. The first stratum can be composed of natural and/or synthetic fibers that do not significantly absorb bodily fluids, and that form an open (i.e., porous) and bulky stratum or web. The first stratum's pore size is preferably greater than that of the second stratum and allows efficient fluid communication and drainage to the second stratum. Synthetic fibers suitable for use in the first stratum include, for example, polyethylene terephthalate (PET), polyethylene, polypropylene, nylon, latex, and rayon fibers. Suitable natural fibers include, for example, cotton, wool, wood pulp, straw, kenaf, and other cellulosic fibers. The basis weight of the first stratum can be in the range from about 20 to about 80 gsm.

The first stratum can further include a binder. Suitable binders include thermoplastic binder fibers such as bicomponent binder fibers (e.g., CELBOND T105 having one half inch in length and 3 denier, commercially available from Kosa, Charlotte, N.C.; Unitika 4080 having 10 mm length and 2 denier, commercially available from Unitika, Japan). In one embodiment, the first stratum includes a mixture of polyethylene terephthalate (PET) fibers (e.g., T224 having one half inch length, 15 denier, and 8 crimp/inch, commercially available from Kosa; DACRON 205NSD having 6 mm length, and 1.5 denier, commercially available from DuPont) and bicomponent binder fibers. In one embodiment, the PET fibers are present in an amount from about 70 to about 90 percent by weight and the bicomponent binder fibers can be present from about 10 to about 30 percent by weight based on the total weight of fibers in the stratum. In one embodiment, the first stratum has a basis weight of about 50 gsm and includes about 80 percent by weight PET fibers and about 20 percent by weight bicomponent fibers based on the total weight of the stratum.

Generally, the greatest rate of liquid acquisition is attained with composites having a first stratum with relatively low density. The formation of low-density strata can be achieved by varying the stratum's components. The performance of the composite is dependent upon a number of factors including fiber length, denier (g/m), crimping (crimps per inch), type of fiber treatment and physical and chemical nature of the fibers of the first stratum. Suitable fibers for inclusion in the first stratum can have a length up to about 1 inch. Suitable fibers include fibers having denier up to about 20 denier. While straight fibers can be advantageously used in the formation of the first stratum, in one embodiment the first stratum includes from about 50 to about 100 percent by weight of total crimped fibers.

Synthetic fibers for inclusion in the first stratum can include polyester fibers having morphologies other than the conventional homogeneous solid fibers noted above. Composites having hollow, deep-grooved, and lobal polyester fibers exhibit advantageous liquid acquisition characteristics. For example, deep-grooved fibers provide strata having low rewet, possibly due in part to improved capillary wicking in the grooves and more rapid liquid evaporation. Hollow fibers provide a composite having enhanced loft compared to composites that include homogeneous solid fibers. Lobal fibers (i.e., fibers having lobal cross-sectional shape) provide composites having a greater resistance to wet collapse compared to solid, round cross-sectioned fiber. For example, lobal polyester fibers are commercially available from Kosa.

In another embodiment, the first stratum is a relatively low basis weight stratum that includes a mixture of matrix fibers (e.g., fluff pulp fibers) and resilient fibers (e.g., crosslinked cellulosic fibers). The basis weight of the stratum can range from about 20 to about 80 gsm. In one embodiment, the stratum includes from about 20 to about 80 percent by weight fluff pulp fibers (e.g., southern pine kraft pulp fibers commercially available from Weyerhaeuser Company under the designation NB416) and from about 80 to about 20 percent by weight crosslinked cellulosic fibers based on the total weight of fibers in the stratum. In another embodiment, the stratum includes from about 30 to about 50 percent by weight fluff pulp fibers and from about 70 to about 50 percent by weight crosslinked fibers based on the total weight of fibers in the stratum. In one embodiment, the stratum has a basis weight of about 40 gsm and includes about 40 percent by weight fluff pulp fibers and about 60 percent by weight crosslinked cellulosic fiber based on the total weight of fibers in the stratum. In another embodiment, the stratum has a basis weight of about 40 gsm and includes about 50 percent by weight fluff pulp fibers and about 50 percent by weight crosslinked cellulosic fibers based on the total weight of fibers in the stratum. In a further embodiment, the stratum has a basis weight of about 20 gsm and includes about 50 percent by weight fluff pulp fibers and about 50 percent by weight crosslinked cellulosic fibers based on the total weight of fibers in the stratum.

The composite's second stratum is an absorbent stratum that can serve as a permanent liquid storage stratum. In general, the second stratum is a fibrous matrix that includes absorbent material. In one embodiment, the fibrous matrix defines voids and passages between the voids, which are distributed throughout the stratum. Absorbent material is located within some of the voids. The absorbent material located in these voids is expandable into the void.

The second stratum is an open and porous stratum characterized as having a stable three-dimensional network of fibers (i.e., fibrous matrix) that create channels or capillaries that serve to rapidly acquire and distribute liquid throughout the stratum, ultimately delivering acquired liquid to the absorbent material that is distributed throughout the stratum.

The second stratum is an open and stable structure that includes a network of capillaries or channels that are effective in acquiring and distributing liquid throughout the stratum. In the stratum, the network of fibers direct fluid throughout the stratum and to absorbent material distributed throughout the stratum. The stratum can include a wet strength agent that serve to stabilize the fibrous structure by providing interfiber bonding. The interfiber bonding assists in providing a stratum having a stable structure in which the stratum's capillaries or channels remain open before, during, and after liquid insult. The stratum's stable structure provides capillaries that remain open after initial liquid insult and that are available for acquiring and distributing liquid on subsequent insults.

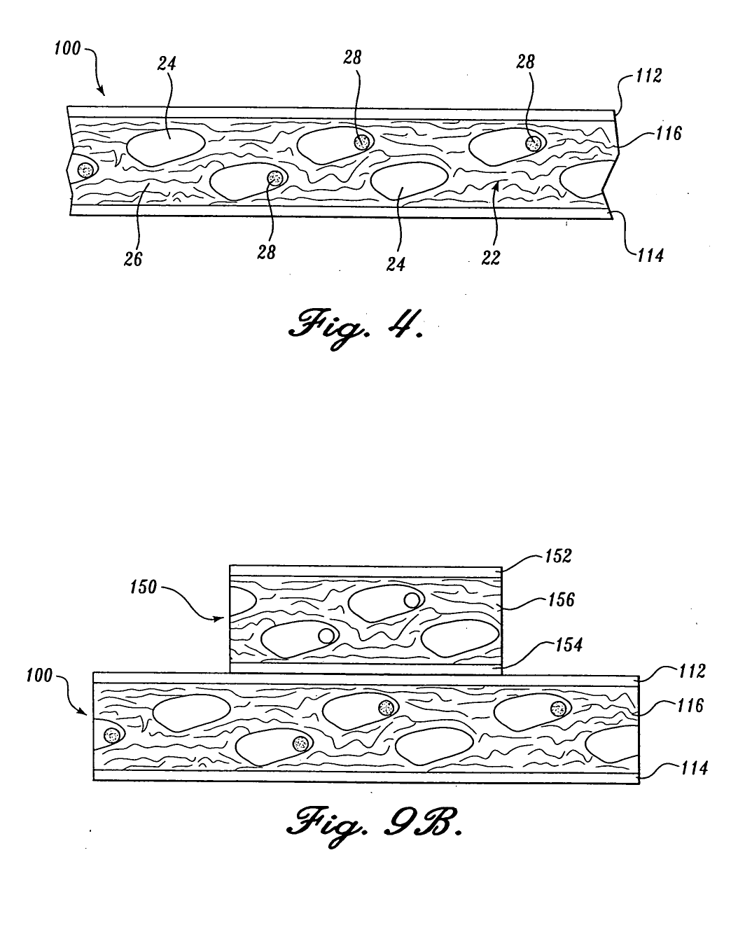

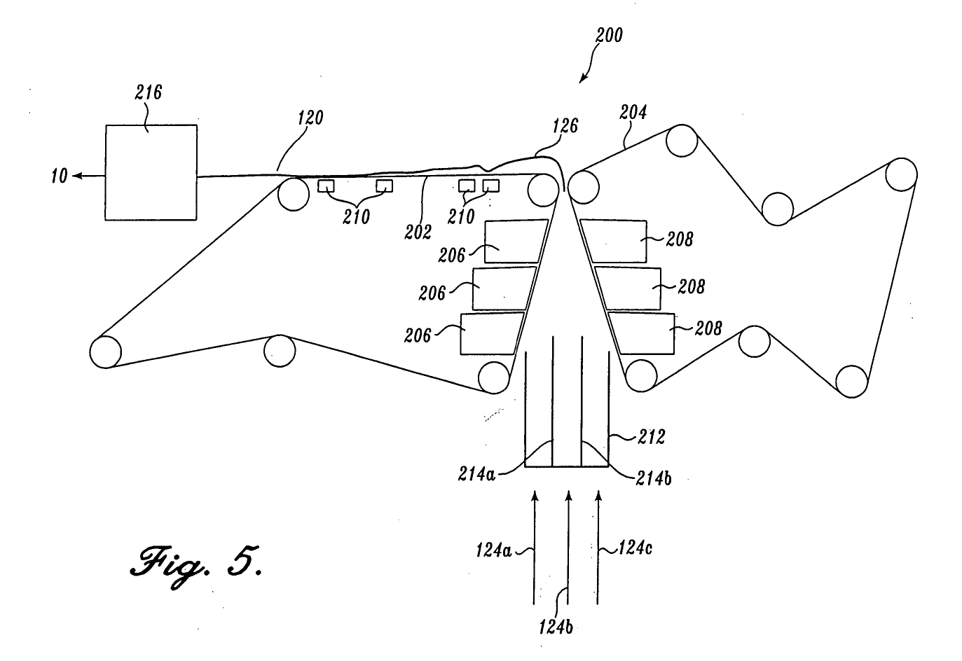

A representative composite of the invention including the absorbent stratum described above is illustrated schematically in FIG. 4. Referring to FIG. 4, representative composite 100 includes first stratum 112, third stratum 114, and second stratum 116, an absorbent stratum that is a fibrous matrix including absorbent material. Stratum 116 includes fibrous regions 22 substantially composed of fibers 26 and defining voids 24. Some voids include absorbent material 28. Voids 24 are distributed throughout stratum 116.

The stratum's voids can be formed by the hydration and swelling of absorbent material (i.e., during wet composite formation) and the subsequent dehydration and decrease in size of the absorbent material (i.e., during wet composite drying). Ultimately, the density of the stratum and composite depends on the extent to which the absorbent material absorbs liquid and swells during the formation of the wet composite, and the conditions and extent to which the wet composite incorporating the swollen absorbent material is dried. Water absorbed by the absorbent material during wet composite formation is removed from the absorbent material, decreasing its size, on drying the wet composite. The dehydration of the swollen absorbent material defines some of the voids in the fibrous stratum.

The second stratum of composite can be an absorbent material-containing stratum as described above and as described in U.S. patent application Ser. No. 09/141,152, international patent application Serial No. PCT/US98/09682, and U.S. patent application Ser. No. 60/046,395, international patent application Serial No. PCT/US99/26560, and U.S. patent application Ser. No. 60/107,998, each expressly incorporated herein by reference in its entirety.

The second stratum can include a fibrous matrix composed of matrix and resilient fibers. Matrix fibers (e.g., fluff pulp fibers) can be present in the stratum in an amount from about 30 to about 80 percent by weight based on the total weight of fibers in the stratum. Resilient fibers (e.g., crosslinked cellulosic fibers) can be present in the stratum in an amount from about 20 to about 70 percent by weight based on the total weight of fibers in the stratum. In one embodiment, the stratum includes about 30 percent by weight matrix fibers and about 70 percent by weight resilient fibers based on the total weight of fibers in the stratum. In another embodiment, the stratum includes about 40 percent by weight matrix fibers and about 60 percent by weight resilient fibers based on the total weight of fibers in the stratum. In a further embodiment, the stratum includes about 50 percent by weight matrix fibers and about 50 percent by weight resilient fibers based on the total weight of fibers in the stratum. In still another embodiment, the stratum includes about 70 percent by weight matrix fibers and about 30 percent by weight resilient fibers based on the total weight of fibers in the stratum. In another embodiment, the stratum includes about 75 percent by weight matrix fibers and about 25 percent by weight resilient fibers based on the total weight of fibers in the stratum.

The second stratum also includes absorbent material in an amount from about 20 to about 80 percent by weight based on the total weight of the stratum. In one embodiment, the stratum includes about 25 percent by weight absorbent material based on the total weight of the stratum. In another embodiment, the stratum includes about 30 percent by weight absorbent material based on the total weight of the stratum. In a further embodiment, the stratum includes about 40 percent by weight absorbent material based on the total weight of the stratum. In still another embodiment, the stratum includes about 45 percent by absorbent material based on the total weight of the stratum. In another embodiment, the stratum includes about 55 percent by weight absorbent material based on the total weight of the stratum. In a further embodiment, the stratum includes about 60 percent by weight absorbent material based on the total weight of the stratum.

The second stratum can further include a wet strength agent. The wet strength agent can be present in the stratum in an amount from about 0.1 to about 0.5 percent by weight based on the total weight of the composite. In one embodiment, the wet strength agent is a polyamide-epichlorohydrin resin commercially available from Hercules under the designation KYMENE.

As noted above, the composite's third stratum can have a composition as described above for the first stratum. In one embodiment, the third stratum has a basis weight of about 20 gsm and includes about 80 percent by weight PET fibers and about 20 percent by weight bicomponent binder fibers based on the total weight of the stratum. In another embodiment, the third stratum has a basis weight of from about 20 to about 40 gsm and includes from about 30 to about 80 percent by weight matrix fibers and from about 70 to about 20 percent by weight resilient fibers based on the total weight of the stratum. In one embodiment, the third stratum has a basis weight of about 30 gsm and includes about 50 percent by weight matrix fibers and about 50 percent by weight resilient fibers based on the total weight of the stratum. In another embodiment, the third stratum has a basis weight of about 30 gsm and includes about 25 percent by weight resilient fibers and about 75 percent by weight matrix fibers (e.g., a refined blend of 75 percent by weight southern pine fluff pulp and 25 percent by weight crosslinked cellulosic fibers) based on the total weight of the stratum.

The basis weight of the composite can vary greatly depending on the intended use of the composite. The composite can have a basis weight in the range from about 150 to about 650 gsm.

The composite of the invention has three strata with the second stratum being a fibrous matrix that includes absorbent material. The compositions of the first and third strata can be varied depending on the composite's intended use. For example, the first and third strata can be composed of synthetic fibers (see Table 1); the first stratum can be composed of synthetic fibers and the third stratum composed of cellulosic fibers (see Table 2); or the first and third strata can be composed of cellulosic fibers (see Table 3).

The compositions of representative composites A-J are summarized in Tables 1-3 below. For these composites, the matrix fiber was kraft southern pine pulp fiber (NB416 commercially available from Weyerhaeuser Company), the synthetic fiber was polyethylene terephthalate (PET) fiber (e.g., T224 or DACRON 205NSD), and the binder fiber was a bicomponent fiber (e.g., CELBOND T105). For the first and third strata, the amount of the specified component included in the stratum is given in weight percent based on the total weight of the stratum. For the second stratum, the amount of absorbent material is given in weight percent based on the total weight of the cellulose-based composite (i.e., weight excludes any synthetic components), and the matrix and crosslinked fiber amounts are in weight percent based on the total weight of fibers in the stratum. In addition to the composites' compositions, Tables 1-3 also summarize the composites' overall basis weight and the basis weights of individual strata. The overall composition of representative composites A-F is summarized in Table 4.

The compositions of representative composites having first and third strata composed of synthetic fibers are summarized in Table 1. The compositions of representative composites having first strata composed of synthetic fibers and third strata composed of cellulosic fibers are summarized in Table 2. The compositions of representative composites having first and third strata composed of cellulosic fibers are summarized in Table 3.

TABLE 1
Representative Composite Compositions.

| | | Second Stratum | | | First Stratum | | | Third Stratum | | |
|---|---|---|---|---|---|---|---|---|---|---|
| | Overall Basis | Absorbent | Matrix | Crosslinked | Basis | Synthetic | Binder | Basis | Synthetic | |
| | Weight | Material | Fiber | Fiber | Weight | Fiber | Fiber | Weight | Fiber (weight | Binder Fiber |
| Composite | (gsm) | (weight %) | (weight %) | (weight %) | (gsm) | (weight %) | (weight %) | (gsm) | %) | (weight %) |
| A | 416 | 45.7 | 50 | 50 | 50 | 80 | 20 | 20 | 80 | 20 |
| B | 394 | 41.2 | 70 | 30 | 50 | 80 | 20 | 20 | 80 | 20 |
| K1 | 427 | 46 | 50 | 50 | 50 | 70¹ | 30 | 20 | 70¹ | 30 |
| K2 | 427 | 46 | 50 | 50 | 50 | 70 | 30 | 20 | 70¹ | 30 |
| K3 | 436 | 45 | 50 | 50 | 50 | 80² | 20 | 30 | 70¹ | 30 |

¹50:20 blend of T224 and DACRON 205NSD
²70:10 blend of T224 and DACRON 205NSD

TABLE 2
Representative Composite Compositions.

| | | Second Stratum | | | First Stratum | | | Third Stratum | | |
|---|---|---|---|---|---|---|---|---|---|---|
| | Overall | Absorbent | Matrix | Crosslinked | Basis | Synthetic | Binder | Basis | | Crosslinked |
| | Basis Weight | Material | Fiber | Fiber | Weight | Fiber | Fiber | Weight | Matrix | Fiber |
| Composite | (gsm) | (weight %) | (weight %) | (weight %) | (gsm) | (weight %) | (weight %) | (gsm) | Fiber (weight %) | (weight %) |
| C | 650 | 60.1 | 30 | 70 | 50 | 80 | 20 | 20 | 30 | 70 |
| D | 375 | 34.9 | 40 | 60 | 50 | 80 | 20 | 20 | 40 | 60 |
| G | 380 | 45 | 50 | 50 | 40 | 80 | 20 | 30 | 50 | 50 |
| L | 390 | 40 | 50 | 50 | 40 | 80² | 20 | 20 | 50 | 50 |

²70:10 blend of T224 and DACRON 205NSD

TABLE 3
Representative Composite Compositions.

| | | Second Stratum | | | First Stratum | | | Third Stratum | | |
|---|---|---|---|---|---|---|---|---|---|---|
| | Overall Basis | Absorbent | Matrix | Crosslinked | Basis | Matrix | Crosslinked | Basis | Matrix | Crosslinked |
| | Weight | Material | Fiber | Fiber | Weight | Fiber | Fiber | Weight | Fiber (weight | Fiber |
| Composite | (gsm) | (weight %) | (weight %) | (weight %) | (gsm) | (weight %) | (weight %) | (gsm) | %) | (weight %) |
| E | 150 | 25 | 40 | 60 | 40 | 40 | 60 | — | — | — |
| F | 374 | 30 | 75 | 25 | 40 | 50 | 50 | — | — | — |
| H | 340 | 55 | 50 | 50 | 20 | 50 | 50 | 30 | 75* | 25* |
| I | 300 | 55 | 50 | 50 | 30 | 75 | 25 | 40 | 50 | 50 |
| J | 245 | 55 | 50 | 50 | 20 | 50 | 50 | — | — | — |
| M1 | 302 | 60 | 50 | 50 | 20 | 50 | 50 | 20 | 75* | 25* |
| M2 | 312 | 58 | 50 | 50 | 20 | 50 | 50 | 30 | 75* | 25* |
| M3 | 322 | 56 | 50 | 50 | 20 | 50 | 50 | 40 | 75* | 25* |

*Refined blend of southern pine fluff pulp and crosslinked cellulosic fibers.

TABLE 4
Representative Composite Overall Composition.

| | Absorbent | | Crosslinked | |
|---|---|---|---|---|
| | Material | Matrix Fiber | Fiber | Other Fibers |
| Composite | (weight %) | (weight %) | (weight %) | (weight %) |
| A | 45.7 | 18.7 | 18.7 | 16.8 |
| B | 41.2 | 28.8 | 12.4 | 17.8 |
| C | 60.1 | 9.7 | 22.5 | 7.7 |
| D | 34.9 | 20.7 | 31.1 | 13.3 |
| E | 25.0 | 30.0 | 45.0 | — |
| F | 30.0 | 49.8 | 20.2 | — |

Some performance characteristics for representative composites K-N are summarized in Table 5 below.

TABLE 5
Representative Composite Performance Characteristics.

| | | | | |
|---|---|---|---|---|
| Basis | Vertical Wicking | Ring | Acquisition | Saturation Capacity |

| | Weight | height | capacity | Crush | Tensile | Elongation | Rate | | BW |
|---|---|---|---|---|---|---|---|---|---|
| Composite | (gsm) | (cm) | (15 min) | (g) | (N/50 mm) | (mm) | (mL/sec) | (g/g) | (gsm) |
| K | 442 | 12.0 | 8.8 | 1142 | 20.0 | 15.0 | 1.51 | 16.43 | 444 |
| L | 382 | 12.2 | 9.2 | 1196 | 19.3 | 13.7 | 1.73 | 16.45 | 381 |
| M | 301 | 14.0 | 12.0 | 1050 | 26.0 | 7.8 | 0.46 | 19.10 | 307 |
| N | 312 | 14.3 | 12.3 | 1065 | 26.0 | 7.0 | 0.62 | 19.10 | 314 |

The composites of the invention can be softened without compromising the composites' liquid wicking properties and its strength (wet and/or dry integrity). In one embodiment, the composite is softened by preferentially softening (e.g., calendering) portions of the composite. In one embodiment, opposing edges of the composite in the composite's machine direction can be softened. In such an embodiment, the central portion of the composite remains unsoftened and the advantageous liquid distribution and strength properties of this portion is preserved unchanged. A representative composite having softened opposing edges of the composite in the composite's machine direction is illustrated in FIG. 11A. An additional benefit of such an embodiment is that the softened opposing edges can be readily folded to provide a C-folded composite as described below. In other embodiments, the composite can be softened by calendering in various patterns including cross-hatched, diagonal, and chevron patterns. Representative composites softened by calendering in cross-hatched, diagonal, and chevron patterns are illustrated in FIGS. 11B-D, respectively.

Fibers are a principal component of the absorbent composite of the invention. Fibers suitable for use in the present invention are known to those skilled in the art and include any fiber from which an absorbent composite can be formed. Suitable fibers include natural and synthetic fibers. Combinations of fibers including combinations of synthetic and natural fibers, and treated and untreated fibers, can also be suitably used in the composite.

The composite of the invention includes resilient fibers. As used herein, the term “resilient fiber” refers to a fiber present in the composite that imparts reticulation to the composite. Generally, resilient fibers provide the composite with bulk and resiliency. The incorporation of resilient fibers into the composite allows the composite to expand on absorption of liquid without structural integrity loss. Resilient fibers also impart softness to the composite. In addition, resilient fibers offer advantages in the composite's formation processes. Because of the porous and open structure resulting from wet composites that include resilient fibers, these composites drain water relatively easily and are therefore dewatered and dried more readily than wet composites that do not include resilient fibers.

Resilient fibers include cellulosic and synthetic fibers. Preferred resilient fibers include chemically stiffened fibers, anfractuous fibers, chemithermomechanical pulp (CTMP), and prehydrolyzed kraft pulp (PHKP).

The term “chemically stiffened fiber” refers to a fiber that has been stiffened by chemical means to increase fiber stiffness under dry and wet conditions. Fibers can be stiffened by the addition of chemical stiffening agents that can coat and/or impregnate the fibers. Stiffening agents include the polymeric wet strength agents including resinous agents such as, for example, polyamide-epichlorohydrin and polyacrylamide resins described below. Fibers can also be stiffened by modifying fiber structure by, for example, chemical crosslinking. Preferably, the chemically stiffened fibers are intrafiber crosslinked cellulosic fibers.

Resilient fibers can include noncellulosic fibers including, for example, synthetic fibers such as polyolefin, polyamide, and polyester fibers. In a preferred embodiment, the resilient fibers include crosslinked cellulosic fibers.

As used herein, the term “anfractuous fiber” refers to a cellulosic fiber that has been chemically treated. Anfractuous fibers include, for example, fibers that have been treated with ammonia.

In addition to resilient fibers, the composite of the invention includes matrix fibers. As used herein, the term “matrix fiber” refers to a fiber that is capable of forming hydrogen bonds with other fibers. Matrix fibers are included in the composite to impart strength to the composite. Matrix fibers include cellulosic fibers such as wood pulp fibers, refined cellulosic fibers, and high surface area fibers such as expanded cellulose fibers. Other suitable cellulosic fibers include cotton linters, cotton fibers, and hemp fibers, among others.

The composite of the present invention preferably includes a combination of resilient and matrix fibers.

Cellulosic fibers are a basic component of the absorbent composite. Although available from other sources, cellulosic fibers are derived primarily from wood pulp. Suitable wood pulp fibers for use with the invention can be obtained from well-known chemical processes such as the kraft and sulfite processes, with or without subsequent bleaching. Pulp fibers can also be processed by thermomechanical, chemithermomechanical methods, or combinations thereof. The preferred pulp fiber is produced by chemical methods. Ground wood fibers, recycled or secondary wood pulp fibers, and bleached and unbleached wood pulp fibers can be used. Softwoods and hardwoods can be used. Details of the selection of wood pulp fibers are well-known to those skilled in the art. These fibers are commercially available from a number of companies, including Weyerhaeuser Company, the assignee of the present invention. For example, suitable cellulose fibers produced from southern pine that are usable with the present invention are available from Weyerhaeuser Company under the designations CF416, NF405, PL416, FR516, and NB416.

Suitable wood pulp fibers can also be pretreated prior to use with the present invention. This pretreatment may include physical treatment, such as subjecting the fibers to steam, or chemical treatment, for example, crosslinking the cellulose fibers using any one of a variety of crosslinking agents. Crosslinking increases fiber bulk and resiliency, and thereby can improve the fibers' absorbency. Generally, crosslinked fibers are twisted or crimped. The use of crosslinked fibers allows the composite to be more resilient, softer, bulkier, and to have enhanced wicking. Suitable crosslinked cellulose fibers produced from southern pine are available from Weyerhaeuser Company under the designation NHB416. Crosslinked cellulose fibers and methods for their preparation are disclosed in U.S. Pat. Nos. 5,437,418 and 5,225,047 issued to Graef et al., expressly incorporated herein by reference.

Crosslinked fibers can be prepared by treating fibers with a crosslinking agent. Suitable cellulose crosslinking agents include aldehyde and urea-based formaldehyde addition products. See, for example, U.S. Pat. Nos. 3,224,926; 3,241,533; 3,932,209; 4,035,147; 3,756,913; 4,689,118; 4,822,453; U.S. Pat. No. 3,440,135, issued to Chung; U.S. Pat. No. 4,935,022, issued to Lash et al.; U.S. Pat. No. 4,889,595, issued to Herron et al.; U.S. Pat. No. 3,819,470, issued to Shaw et al.; U.S. Pat. No. 3,658,613, issued to Steiger et al.; and U.S. Pat. No. 4,853,086, issued to Graef et al., all of which are expressly incorporated herein by reference in their entirety. Cellulose fibers have also been crosslinked by carboxylic acid crosslinking agents including polycarboxylic acids. U.S. Pat. Nos. 5,137,537; 5,183,707; and 5,190,563, describe the use of C2-C9 polycarboxylic acids that contain at least three carboxyl groups (e.g., citric acid and oxydisuccinic acid) as crosslinking agents.

Suitable urea-based crosslinking agents include methylolated ureas, methylolated cyclic ureas, methylolated lower alkyl substituted cyclic ureas, methylolated dihydroxy cyclic ureas, dihydroxy cyclic ureas, and lower alkyl substituted cyclic ureas. Specific preferred urea-based crosslinking agents include dimethylol urea (DMU, bis[N-hydroxymethyl]urea), dimethylolethylene urea (DMEU, 1,3-dihydroxymethyl-2-imidazolidinone), dimethyloldihydroxyethylene urea (DMDHEU, 1,3-dihydroxymethyl-4,5-dihydroxy-2-imidazolidinone), dimethyldihydroxy urea (DMDHU), dihydroxyethylene urea (DHEU, 4,5-dihydroxy-2-imidazolidinone), and dimethyldihydroxyethyleneu urea (DMeDHEU, 4,5-dihydroxy-1,3-dimethyl-2-imidazolidinone).

Suitable polycarboxylic acid crosslinking agents include citric acid, tartaric acid, malic acid, succinic acid, glutaric acid, citraconic acid, itaconic acid, tartrate monosuccinic acid, and maleic acid. Other polycarboxylic acids crosslinking agents include polymeric polycarboxylic acids such as poly(acrylic acid), poly(methacrylic acid), poly(maleic acid), poly(methylvinylether-co-maleate) copolymer, poly(methylvinylether-co-itaconate) copolymer, copolymers of acrylic acid, and copolymers of maleic acid. The use of polymeric polycarboxylic acid crosslinking agents such as polyacrylic acid polymers, polymaleic acid polymers, copolymers of acrylic acid, and copolymers of maleic acid is described in U.S. Pat. No. 5,998,511. Mixtures or blends of crosslinking agents may also be used.

The crosslinking agent can include a catalyst to accelerate the bonding reaction between the crosslinking agent and cellulose fiber. Suitable catalysts include acidic salts, such as ammonium chloride, ammonium sulfate, aluminum chloride, magnesium chloride, and alkali metal salts of phosphorous-containing acids.

Although not to be construed as a limitation, examples of pretreating fibers include the application of surfactants or other liquids which modify the surface chemistry of the fibers. Other pretreatments include incorporation of antimicrobials, pigments, dyes and densification or softening agents. Fibers pretreated with other chemicals, such as thermoplastic and thermosetting resins also may be used. Combinations of pretreatments also may be employed. Similar treatments can also be applied after the composite formation in post-treatment processes.

Cellulosic fibers treated with particle binders and/or densification/softness aids known in the art can also be employed in accordance with the present invention. The particle binders serve to attach other materials, such as cellulosic fiber superabsorbent polymers, as well as others, to the cellulosic fibers. Cellulosic fibers treated with suitable particle binders and/or densification/softness aids and the process for combining them with cellulose fibers are disclosed in the following U.S. patents: (1) Pat. No. 5,543,215, entitled “Polymeric Binders for Binding Particles to Fibers”; (2) Pat. No. 5,538,783, entitled “Non-Polymeric Organic Binders for Binding Particles to Fibers”; (3) Pat. No. 5,300,192, entitled “Wet Laid Fiber Sheet Manufacturing With Reactivatable Binders for Binding Particles to Binders”; (4) Pat. No. 5,352,480, entitled “Method for Binding Particles to Fibers Using Reactivatable Binders”; (5) Pat. No. 5,308,896, entitled “Particle Binders for High-Bulk Fibers”; (6) Pat. No. 5,589,256, entitled “Particle Binders that Enhance Fiber Densification”; (7) Pat. No. 5,672,418, entitled “Particle Binders”; (8) Pat. No. 5,607,759, entitled “Particle Binding to Fibers”; (9) Pat. No. 5,693,411, entitled “Binders for Binding Water Soluble Particles to Fibers”; (10) Pat. No. 5,547,745, entitled “Particle Binders”; (11) Pat. No. 5,641,561, entitled “Particle Binding to Fibers”; (12) Pat. No. 5,308,896, entitled “Particle Binders for High-Bulk Fibers”; (13) Pat. No. 5,498,478, entitled “Polyethylene Glycol as a Binder Material for Fibers”; (14) Pat. No. 5,609,727, entitled “Fibrous Product for Binding Particles”; (15) Pat. No. 5,571,618, entitled “Reactivatable Binders for Binding Particles to Fibers”; (16) Pat. No. 5,447,977, entitled “Particle Binders for High Bulk Fibers”; (17) Pat. No. 5,614,570, entitled “Absorbent Articles Containing Binder Carrying High Bulk Fibers; (18) Pat. No. 5,789,326, entitled “Binder Treated Fibers”; and (19) Pat. No. 5,611,885, entitled “Particle Binders”; each expressly incorporated herein by reference.

Modified cellulosic fibers useful in the invention include rayon and cellulose acetate fibers.

In addition to natural fibers, synthetic fibers including polymeric fibers, such as polyolefin, polyamide, polyester, polyvinyl alcohol, polyvinyl acetate fibers, can also be used in the absorbent composite of the present invention. Suitable synthetic fibers include, for example, polyethylene terephthalate, polyethylene, polypropylene, and nylon fibers. Other suitable synthetic fibers include those made from thermoplastic polymers, cellulosic and other fibers coated with thermoplastic polymers, and multicomponent fibers in which at least one of the components includes a thermoplastic polymer. Single and multicomponent fibers can be manufactured from polyester, polyethylene, polypropylene, and other conventional thermoplastic fibrous materials. Single and multicomponent fibers are commercially available. Suitable bicomponent fibers include CELBOND fibers available from Kosa and Unitika 4080 fibers available from Unitika. The absorbent composite can also include combinations of natural and synthetic fibers.

To enhance liquid absorption, acquisition, distribution, and storage, the composite of the invention includes a stratum that includes absorbent material. As used herein, the term “absorbent material” refers to a material that absorbs liquid and that generally has an absorbent capacity greater than the cellulosic fibrous component of the composite. Preferably, the absorbent material is a water swellable, generally water insoluble polymeric material capable of absorbing at least about 5, desirably about 20, and preferably about 100 times or more its weight in saline (e.g., 0.9 percent saline). The absorbent material can be swellable in the dispersion medium utilized in the method for forming the composite. In one embodiment, the absorbent material is untreated and swellable in the dispersion medium. In another embodiment, the absorbent material is an absorbent material that is resistant to absorbing water during the composite formation process. Such absorbent materials that are resistant to absorption include coated and chemically modified absorbent materials.

The amount of absorbent material present in the composite can vary greatly depending on the composite's intended use. The amount of absorbent material present in an absorbent article such as an absorbent core for an infant's diaper can be from about 20 to about 70 weight percent by weight based on the total weight of the core.

The absorbent material may include natural materials such as agar, pectin, and guar gum, and synthetic materials, such as synthetic hydrogel polymers. Synthetic hydrogel polymers include, for example, carboxymethyl cellulose, alkaline metal salts of polyacrylic acid, polyacrylamides, polyvinyl alcohol, ethylene maleic anhydride copolymers, polyvinyl ethers, hydroxypropyl cellulose, polyvinyl morpholinone, polymers and copolymers of vinyl sulphonic acid, polyacrylates, polyacrylamides, and polyvinyl pyridine among others. In one embodiment, the absorbent material is a superabsorbent material. As used herein, a “superabsorbent material” refers to a polymeric material that is capable of absorbing large quantities of fluid by swelling and forming a hydrated gel (i.e., a hydrogel). In addition to absorbing large quantities of fluids, superabsorbent polymers can also retain significant amounts of bodily fluids under moderate pressure.

Superabsorbent polymers generally fall into three classes: starch graft copolymers, crosslinked carboxymethylcellulose derivatives, and modified hydrophilic polyacrylates. Examples of such absorbent polymers include hydrolyzed starch-acrylonitrile graft copolymers, neutralized starch-acrylic acid graft copolymers, saponified acrylic acid ester-vinyl acetate copolymers, hydrolyzed acrylonitrile copolymers or acrylamide copolymers, modified crosslinked polyvinyl alcohol, neutralized self-crosslinking polyacrylic acids, crosslinked polyacrylate salts, carboxylated cellulose, and neutralized crosslinked isobutylene-maleic anhydride copolymers.

Superabsorbent polymers are available commercially, for example, polyacrylates from Clariant of Portsmouth, Va. These superabsorbent polymers come in a variety of sizes, morphologies and absorbent properties (available from Clariant under trade designations such as IM 3500 and IM 3900). Other superabsorbent particles are marketed under the trademarks SANWET (supplied by Sanyo Kasei Kogyo Kabushiki Kaisha), and SXM77 (supplied by Stockhausen of Greensboro, N.C.). Other superabsorbent polymers are described in U.S. Pat. No. 4,160,059; U.S. Pat. No. 4,676,784; U.S. Pat. No. 4,673,402; U.S. Pat. No. 5,002,814; U.S. Pat. No. 5,057,166; U.S. Pat. No. 4,102,340; and U.S. Pat. No. 4,818,598, all expressly incorporated herein by reference. Products such as diapers that incorporate superabsorbent polymers are described in U.S. Pat. No. 3,699,103 and U.S. Pat. No. 3,670,731.

Suitable superabsorbent polymers useful in the absorbent composite of the present invention include superabsorbent polymer particles and superabsorbent polymer fibers.

In one embodiment, the absorbent composite of the present invention includes a superabsorbent material that that swells relatively slowly for the purposes of composite manufacturing and yet swells at an acceptable rate so as not to adversely affect the absorbent characteristics of the composite or any construct containing the composite.

Composite wet and dry strength can be increased by the incorporation of a binder. Alternatively, for composites that do not include a binder, composite integrity can be achieved through densification.

As noted above, the composites of the invention can include a binder. Suitable binders include, but are not limited to, cellulosic and synthetic fibrous materials, bonding agents, soluble bonding mediums, and wet strength agents as described below. In one embodiment, the binder includes a bicomponent binding fiber, such as CELBOND (Kosa) and D-271P® (DuPont) fibers. In another embodiment, the binder includes a soluble binding medium, such as cellulose acetate used in combination with the solvent triacetin and/or triethyl citrate.

As used herein, the term “binder” refers to a system that is effective in mechanically intertwining or bonding the materials within a stratum, or bonding the materials of adjacent strata. Suitable binders can include, but are not limited to, bonding agents such as thermoplastic and thermosetting materials, soluble bonding mediums used in combination with solvents, and wet strength agents.

Bonding agents useful in the binder in accordance with the present invention are those materials that (a) are capable of being combined with and dispersed throughout a web of fibers, (b) when activated, are capable of coating or otherwise adhering to the fibers or forming a binding matrix, and (c) when deactivated, are capable of binding at least some of the fibers together. The use of bonding agents with cellulose fiber webs is disclosed in U.S. patent application Ser. No. 08/337,642, filed Nov. 10, 1994, entitled “Densified Cellulose Fiber Pads and Methods of Making the Same,” expressly incorporated herein by reference.

Suitable bonding agents include thermoplastic materials that are activated by melting at temperatures above room temperature. When these materials are melted, they will coat at least portions of the cellulose fibers with which they are combined. When the thermoplastic bonding agents are deactivated by cooling to a temperature below their melt point, and preferably no lower than room temperature, the bonding agent will, upon solidifying from the melted state, cause the cellulose fibers to be bound in a matrix.

Thermoplastic materials can be combined with the fibers in the form of particles, emulsions, or as fibers. Suitable fibers can include those made from thermoplastic polymers, cellulosic or other fibers coated with thermoplastic polymers, and multicomponent fibers in which at least one of the components of the fiber comprises a thermoplastic polymer. Single and multicomponent fibers are manufactured from polyester, polyethylene, polypropylene, and other conventional thermoplastic fiber materials. The same thermoplastics can be used in particulate or emulsion form. Many single-component fibers are readily commercially available. Suitable multicomponent fibers include CELBOND fibers available from Kosa. One crimped polymer-based binder fiber is Kosa copolyolefin bicomponent fiber, commercially available under the tradename CELBOND from Kosa, type 255, lot 33865A, having a detex of about 3.3, a denier of about 3.0, and a fiber length of about 6.4 mm. Suitable coated fibers can include cellulose fibers coated with latex or other thermoplastics, as disclosed in U.S. Pat. No. 5,230,959, issued Jul. 27, 1993, to Young et al., and U.S. Pat. No. 5,064,689, issued Nov. 12, 1991, to Young et al. The thermoplastic fibers are preferably combined with the cellulose fibers before or during the forming process. When used in particulate or emulsion form, the thermoplastics can be combined with the cellulose fibers before, during, or after the forming process.

Other suitable thermoplastic bonding agents include ethylene vinyl alcohol, polyvinyl acetate, acrylics, polyvinyl acetate acrylate, polyvinyl dichloride, ethylene vinyl acetate, ethylene vinyl chloride, polyvinyl chloride, styrene, styrene acrylate, styrene butadiene, styrene acrylonitrile, butadiene acrylonitrile, acrylonitrile butadiene styrene, ethylene acrylic acid, urethanes, polycarbonate, polyphenylene oxide, and polyimides.

Thermosetting materials also serve as bonding agents for use in the present invention. Typical thermosetting materials are activated by heating to elevated temperatures at which crosslinking occurs. Alternatively, a resin can be activated by combining it with a suitable crosslinking catalyst before or after it has been applied to the cellulosic fiber. Thermosetting resins can be deactivated by allowing the crosslinking process to run to completion or by cooling to room temperature, at which point crosslinking ceases. When crosslinked, it is believed that the thermosetting materials form a matrix to bond the cellulose fibers. It is contemplated that other types of bonding agents can also be employed, for example, those that are activated by contact with steam, moisture, microwave energy, and other conventional means of activation.

Thermosetting bonding agents suitable for the present invention include phenolic resins, polyvinyl acetates, urea formaldehyde, melamine formaldehyde, and acrylics. Other thermosetting bonding agents include epoxy, phenolic, bismaleimide, polyimide, melamine formaldehyde, polyester, urethanes, and urea.

These bonding agents are normally combined with the fibers in the form of an aqueous emulsion. They can be combined with the fibers during the laying process. Alternatively, they can be sprayed onto a loose web after it has been formed.

As noted above, the binder utilized in accordance with the present invention can also be a soluble bonding medium that can be incorporated with the pulped cellulosic fibers, either in fiber form, or as particles or granules. If desired, the bonding medium can also be coated onto solvent-insoluble fibers, such as cellulosic fibers, which can then be distributed throughout the matrix of fibers making up each of the strata of the present invention. It is presently preferred that the bonding medium comprise a fiber and be mixed with the components of each stratum prior to the formation of the absorbent. The use of soluble bonding mediums with cellulose fiber webs is disclosed in U.S. Pat. No. 5,837,627, entitled “Fibrous Web Having Improved Strength and Method of Making the Same,” expressly incorporated herein by reference.

The solvents employed in accordance with the present invention must of course be capable of partially solubilizing the bonding medium as described above. The solvents must be able to partially dissipate or migrate from the surface of the bonding medium to allow the bonding medium to resolidify after partial solubilization. Nonvolatile solvents may be dissipated in most part by absorption into the bonding medium. It is preferred that the solvent be of limited volatility, so that little or no solvent will be lost to the atmosphere. By limited volatility it is meant that the solvent has a vapor pressure of 29 kPa or less at 25° C. Using a solvent of limited volatility may mitigate precautions usually necessary to control volatiles, and reduces the amount of solvent required to partially solubilize the bonding medium. In addition, use of solvents of limited volatility may eliminate the attendant processing problems encountered with volatile solvents, many of which are flammable and must be handled with care. The use of solvents of limited volatility may also reduce environmental problems. Furthermore, it is desirable for solvents to be nontoxic and capable of being dissipated from the surface of the bonding medium without adversely affecting the overall strength of the bonding medium.

Preferred bonding mediums and solvents of limited volatility include cellulose acetate and solvents including triacetin, propane diol diacetate, propane diol, diproprionate, propane diol dibutyrate, triethyl citiate, dimethyl phthalate, and dibutyl phthalate; cellulose nitrate and triacetin; cellulose butyrate and triacetin; vinyl chloride/vinyl acetate copolymer and triacetin; and cellulose fibers coated with polyvinyl acetate and triacetin.

Of the several bonding mediums listed, cellulose acetate is the most preferred. During manufacture of cellulose acetate fibers, a finish is usually applied to the fibers. Many times this finish is in the form of an oil. The presence of the finish sometimes detracts from the performance of a bonding medium. The presence of a finish may adversely affect the development as well as the strength of the bonds. It has been found that, when the bonding fibers are as straight as possible, as opposed to curled or kinked, they provide more contact points with the cellulosic fibers, and thus the final web will develop better strength. Similarly, when the bonding fibers are as long as is reasonably possible, the strength of the final web is increased. In addition to the foregoing, cellulose ethers and other cellulose esters may also be used as bonding medium. Acetylated pulp fibers may also be used as bonding medium and may be substituted with any number of acetyl groups. A preferred degree of substitution (D.S.) would be 2 to 3, and a most preferred D.S. would be 2.4.

The solvents used in combination with the bonding medium can be added in varying amounts. Strength is adversely affected if too little or too much solvent is added. At a cellulose acetate/pulp weight ratio of 10:90, it has been found that the solvents, and particularly triacetin, provide good strength when added in amounts ranging from 6 percent to 17 percent, and most preferably in the range of 9 percent to 14 percent, based on the weight of pulp fiber present.

The preferred forms of the solvents propane diol diacetate, dipropionate, and dibutyrate are the 1, 2 and 1, 3 forms. Other suitable solvents that work in accordance with present invention are butyl phthalyl butyl glycolate, N-cyclohexyl-p-toluenesulfonamide, diamyl phthalate, dibutyl phthalate, dibutyl succinate, dibutyl tartrate, diethylene glycol dipropionate, di-(2-ethoxyethyl) adipate, di-(2-ethoxyethyl) phthalate, diethyl adipate, diethyl phthalate, diethyl succinate, diethyl tartrate, di-(2-methoxyethyl) adipate, di-(2-methoxyethyl) phthalate, dimethyl phthalate, dipropyl phthalate, ethyl o-benzoylbenzoate, ethyl phthalyl ethyl glycolate, ethylene glycol diacetate, ethylene glycol dibutyrate, ethylene glycol dipropionate, methyl o-benzoylbenzoate, methyl phthalyl ethyl glycolate, N-o and p-tolylethylsulfonamide, o-tolyl p-toluenesulfonate, tributyl citrate, tributyl phosphate, tributyrin, triethylene glycol diacetate, triethylene glycol dibutyrate, triethylene glycol dipropionate, and tripropionin.

The binder useful in the absorbent composite of the invention can also include polymeric agents that can coat or impregnate cellulosic fibers. These wet strength agents provide increased strength to the absorbent composite and enhance the composites wet integrity. In addition to increasing the composites wet strength, the wet strength agent can assist in binding the absorbent material, for example, superabsorbent material, in the composite's fibrous matrix.

Suitable wet strength agents include cationic modified starch having nitrogen-containing groups (e.g., amino groups) such as those available from National Starch and Chemical Corp., Bridgewater, N.J.; latex; wet strength resins such as polyamide-epichlorohydrin resin (e.g., KYMENE 557LX, Hercules, Inc., Wilmington, Del.), polyacrylamide resin (described, for example, in U.S. Pat. No. 3,556,932 issued Jan. 19, 1971 to Coscia et al.; also, for example, the commercially available polyacrylamide marketed by American Cyanamid Co., Stanford, Conn., under the trade name PAREZ 631 NC); urea formaldehyde and melamine formaldehyde resins, and polyethylenimine resins. A general discussion on wet strength resins utilized in the paper field, and generally applicable in the present invention, can be found in TAPPI monograph series No. 29, “Wet Strength in Paper and Paperboard”, Technical Association of the Pulp and Paper Industry (New York, 1965).

Generally, the wet strength agent is present in the composition in an amount from about 0.01 to about 2 weight percent, preferably from about 0.1 to about 1 weight percent, and more preferably from about 0.3 to about 0.7 weight percent, based on the total weight of the composite. In a preferred embodiment, the wet strength agent useful in the composite of the present invention is a polyamide-epichlorohydrin resin such as commercially available from Hercules, Inc. under the designation KYMENE. The wet and dry tensile strength of an absorbent composite formed in accordance with the present invention will generally increase with an increasing the amount of wet strength agent.

Other binders could also include the use of scrim and/or continuous fiber filaments.

Additives can also be incorporated into the composite formed in accordance with the present invention during absorbent formation. The advantage of incorporating the additives during the absorbent formation is that they will also be attached to the absorbent matrix. This provides a significant advantage in that the additives can be dispersed and retained throughout the matrix where desired. For example, the additives may be evenly dispersed and retained throughout the matrix. Additives that can be incorporated into the matrix include absorbent capacity enhancing materials such as superabsorbent polymers, adsorbents such as clays, zeolites, and activated carbon, brighteners such as titanium oxide, and odor absorbents such as sodium bicarbonate.

In one embodiment, the absorbent composite is a densified composite. Densification methods useful in producing the densified composites of the present invention are well known to those in the art. See, for example, U.S. Pat. No. 5,547,541 and patent application Ser. No. 08/859,743, filed May 21, 1997, entitled “Softened Fibers and Methods of Softening Fibers,” assigned to Weyerhaeuser Company, both expressly incorporated herein by reference. Post dryer densified absorbent composites of this invention generally have a density from about 0.1 to about 0.5 g/cm³, and preferably about 0.15 g/cm³. Predryer densification can also be employed. Preferably, the absorbent composite is densified by either a heated or room temperature calender roll method. See, for example, U.S. Pat. Nos. 5,252,275 and 5,324,575, each expressly incorporated herein by reference.

In another aspect of the present invention, methods for forming the composite described above are provided. The composite can be formed by wet-laid and foam-forming processes. A representative example of a wet-laid process is described in U.S. Pat. No. 5,300,192, issued Apr. 5, 1994, entitled “Wet-Laid Fiber Sheet Manufacturing with Reactivatable Binders for Binding Particles to Fibers”, expressly incorporated herein by reference. Wet-laid processes are also described in standard texts, such as Casey, Pulp and Paper, 2nd edition, 1960, Volume II, Chapter VIII —Sheet Formation. Representative foam processes useful in forming the composite of the present invention are known in the art and include those described in U.S. Pat. Nos. 3,716,449; 3,839,142; 3,871,952; 3,937,273; 3,938,782; 3,947,315; 4,166,090; 4,257,754; and 5,215,627, assigned to Wiggins Teape and related to the formation of fibrous materials from foamed aqueous fiber suspensions, and “The Use of an Aqueous Foam as a Fiber-Suspending Medium in Quality Papermaking,” Foams, Proceedings of a Symposium organized by the Society of Chemical Industry, Colloid and Surface Chemistry Group, R. J. Akers, Ed., Academic Press, 1976, which describes the Radfoam process, all expressly incorporated herein by reference.

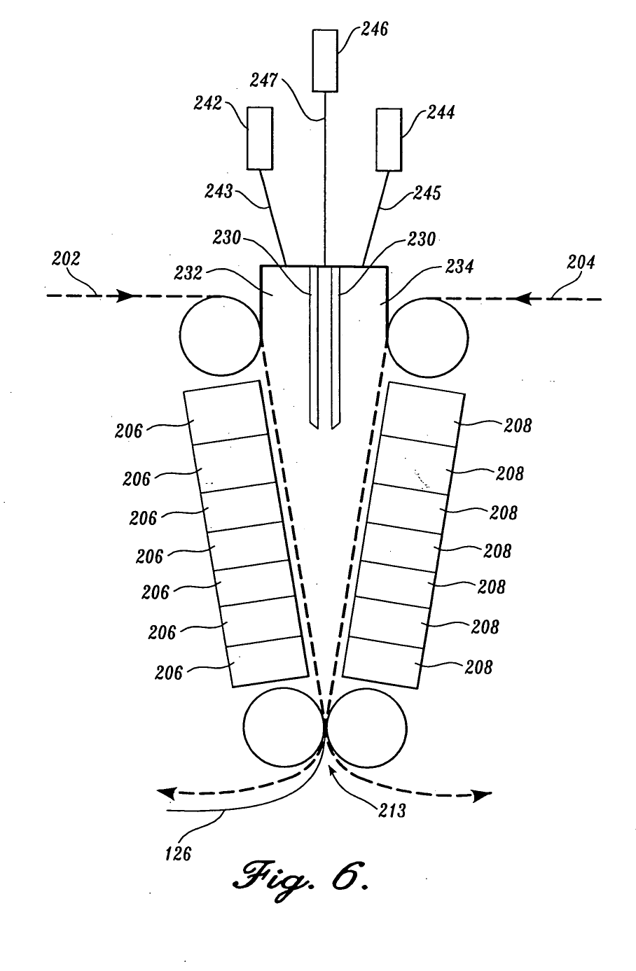

The composite of the invention can be formed by devices and processes that include a twin-wire configuration (i.e., twin-forming wires). A representative twin-wire machine for forming composites of the invention is shown in FIG. 5. Referring to FIG. 5, machine 200 includes twin-forming wires 202 and 204 onto which the composite's components are deposited. Basically, fibrous slurry 124 (which may include slurries 124A, 124B, and 124C) is introduced into headbox 212 and deposited onto forming wires 202 and 204 at the headbox exit. Vacuum elements 206 and 208 dewater the fibrous slurries deposited on wires 202 and 204, respectively, to provide partially dewatered webs that exit the twin-wire portion of the machine as partially dewatered web 126. Web 126 continues to travel along wire 202 and continues to be dewatered by additional vacuum elements 210 to provide wet composite 120 which is then dried by drying means 216 to provide composite 10.

Representative composites formed by the twin-wire method of the present invention are shown in FIGS. 1, 2, and 4. The composites can be formed from multilayered inclined formers or twin-wire formers with sectioned headboxes. These methods can provide stratified composites with strata having specifically designed properties and containing components to attain composites having desired properties.

Referring to FIGS. 1, 3, and 5, composite 10 having strata 12, 14, and 16 can be formed by machine 200. For composites in which the strata comprise the same components, a single fiber furnish 124 is introduced into headbox 212. For forming composites having strata comprising different components, headbox 212 includes one or more baffles (or dividers) 214 (e.g., 214a and 214b) for the introduction of fiber furnishes (e.g., 124a, 124b, and 124c) having different compositions. In such a method, the upper and lower strata (i.e., first and third strata) can be formed to include different components and have different basis weights and properties.

In one embodiment, the composite is formed by a foam-forming method using the components described above. For foam-forming methods, the fibrous furnishes are foam furnishes and include a surfactant. In one embodiment, the foam-forming method is practiced on a twin-wire former.

The method can provide composites having three strata. A representative composite having three strata includes a first stratum formed from fibers. (e.g., synthetic fibers and binder fibers); an intermediate stratum formed from cellulosic fibers and other absorbent material such as superabsorbent material; and a third stratum also formed from fibers (e.g., synthetic and/or cellulosic fibers). The method of the invention is versatile in that such a composite can have relatively distinct and discrete strata or, alternatively, have gradual transition zones from stratum-to-stratum.

A representative method for forming a fibrous web having an intermediate stratum (i.e., a composite having three strata) generally includes the following steps:
(a) forming a first fibrous furnish comprising fibers in an aqueous dispersion medium;
(b) forming a second fibrous furnish comprising fibers in an aqueous dispersion medium;
(c) moving a first foraminous element (e.g., a forming wire) in a first path;
(d) moving a second foraminous element in a second path;
(e) passing the first furnish into contact with the first foraminous element moving in a first path;
(f) passing the second furnish into contact with the second foraminous element moving in the second path;
(g) passing a third material between the first and second furnishes such that the third material does not contact either of the first or second foraminous elements; and
(h) forming a fibrous web from the first and second furnishes and third material by withdrawing liquid from the furnishes through the first and second foraminous elements.

As noted above, the method is suitably carried out on a twin-wire former; in one embodiment, a vertical former; and in another embodiment, a vertical downflow twin-wire former. In the vertical former, the paths for the foraminous elements are substantially vertical.

A representative vertical downflow twin-wire former useful in practicing the method of the invention is illustrated in FIG. 6. Referring to FIG. 6, the former includes a vertical headbox assembly having a former with a closed first end (top), closed first and second sides and an interior volume. A second end (bottom) of the former is defined by moving first and second foraminous elements, 202 and 204, and forming nip 213. The interior volume defined by the former's closed first end, closed first and second sides, and first and second foraminous elements includes an interior structure 230 extending from the former first end and toward the second end. The interior structure defines a first volume 232 on one side thereof and a second volume 234 on the other side thereof. The former further includes supply 242 and means 243 for introducing a first furnish into the first volume, supply 244 and means 245 for introducing a second furnish into the second volume, and supply 246 and means 247 for introducing a third material into the interior structure. Means for withdrawing liquid (e.g., suction boxes 206 and 208) from the first and second slurries through the foraminous elements to form a web are also included in the headbox assembly.

In the method, the twin-wire former includes a means for introducing at least a third material through the interior structure. In one embodiment, the introducing means include at least a first plurality of conduits having a first effective length. A second plurality of conduits having a second effective length different from the first length may also be used. More than two sets of conduits can also be used.

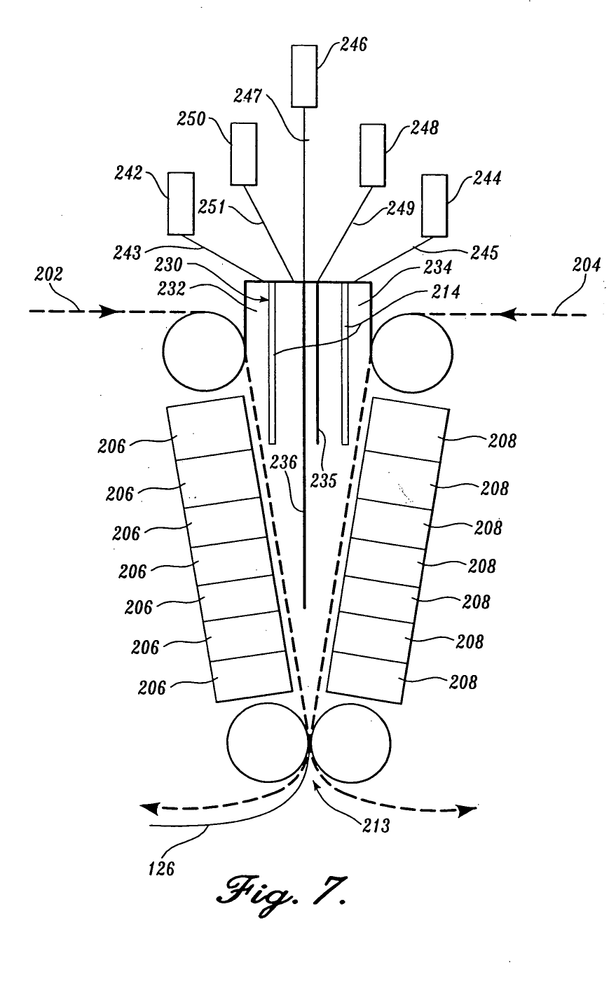

Another representative vertical downflow twin-wire former useful in practicing the method of the invention is illustrated in FIG. 7. Referring to FIG. 7, the former includes a vertical headbox assembly having an interior volume defined by the former's closed first end, closed first and second sides, and first and second foraminous elements, 202 and 204, and includes an interior structure 230 extending from the former first end and toward the second end. In this embodiment, interior structure 230 includes plurality of conduits 235 and 236, and optional divider walls 214a and 214b.

The interior structure defines a first volume 232 on one side thereof and a second volume 234 on the other side thereof. The former further includes supply 242 and means 243 for introducing a first furnish into the first volume, supply 244 and means 245 for introducing a second furnish into the second volume, supply 246 and means 247 for introducing a third material into plurality of conduits 236, supply 248 and means 249 for introducing a third material into plurality of conduits 235, and supply 250 and means 251 for introducing another material, such as a foam slurry, within the volume defined by walls 214.

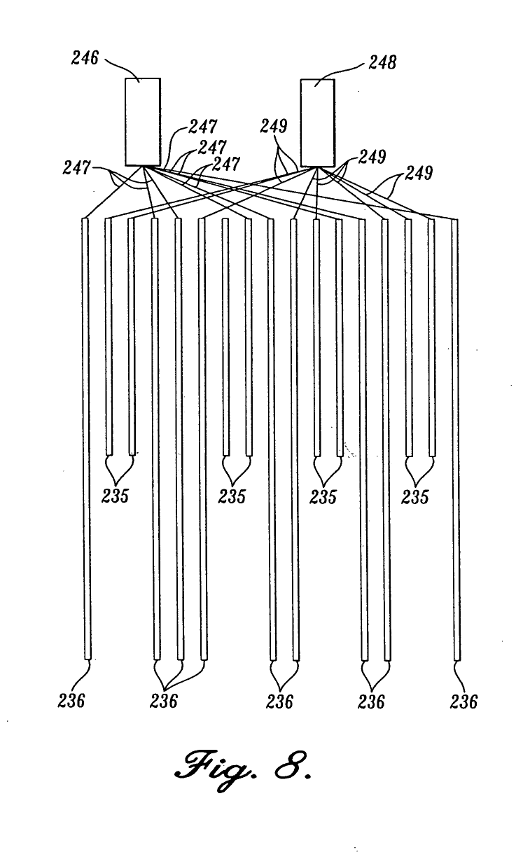

Plurality of conduits 235 can have an effective length different from plurality of conduits 236. The third material can be introduced through conduits 235 and 236, or, alternatively, a third material can be introduced through conduits 235 and a fourth material can be introduced through conduits 236. Preferably, the ends of conduits 235 and 236 terminate at a position beyond where the suction boxes begin withdrawing foam from the slurries in contact with the foraminous elements (i.e., beyond the point where web formation begins). Plurality of conduits 235 and 236 can be moved in a first dimension toward and away from nip 213, and also in a second dimension substantially perpendicular to the first, closer to one forming wire or the other. Representative plurality of conduits 235 and 236 are illustrated in FIG. 8.

Generally, the former's interior structure (i.e., structure 230 in FIGS. 6 and 7) is positioned with respect to the foraminous elements such that material introduced through the interior structure will not directly contact the first and second foraminous elements. Accordingly, material is introduced through the interior structure between the first and second slurries after the slurries have contacted the foraminous elements and withdrawal of foam and liquid from those slurries has commenced. Such a configuration is particularly advantageous for introducing absorbent material (e.g., superabsorbent materials) and for forming stratified structures in which the third material is a fiber furnish (e.g., a fibrous furnish including absorbent material). Depending upon the nature of the composite to be formed, the first and second furnishes may be the same as, or different from, each other and from the third material.

The method can also include utilizing a plurality of distinct conduits, the conduits being of at least two different lengths, for introducing the third material into the headbox. The method can also be utilized in headboxes having dividing walls that extend part of the length of the conduits toward the headbox exit.

The means for introducing first and second furnishes into the first and second volumes can include any conventional type of conduit, nozzle, orifice, header, or the like. Typically, these means include a plurality of conduits are provided disposed on the first end of the former and facing the second end.

The means for withdrawing liquid from the first and second furnishes through the foraminous elements to form a web on the foraminous elements are also included in the headbox assembly. The means for withdrawing liquid can include any conventional means for that purpose, such as suction rollers, pressing rollers, or other conventional structures. In one embodiment, first and second suction box assemblies are provided and mounted on the opposite sides of the interior structure from the foraminous elements (see boxes 206 and 208 in FIGS. 5-7).

Absorbent material can be introduced into the headbox of a former as the third material. In one embodiment, absorbent material can be introduced as a component in a fibrous slurry. In this embodiment, absorbent material can be combined with a fibrous slurry (e.g., a blend of matrix and resilient fibers) and introduced into the headbox. Referring to FIG. 5, fibrous slurry including absorbent material identified as 124a can be introduced into headbox 212 between dividers 214a and 214b. Referring to FIG. 6, a fibrous slurry including absorbent material can be introduced to interior structure 230 through conduit 247 from supply 246. In another embodiment, absorbent material can be introduced into a former's headbox as a solid suspension in an aqueous dispersion medium. Referring to FIGS. 7 and 8, an absorbent material suspension can be introduced through conduits 235 and/or 236, whereupon exiting the conduits the absorbent material encounters fibrous material that has also been introduced into the headbox.

Absorbent material can be introduced into the headbox as a dry particle or as a liquid suspension in an aqueous medium, preferably chilled (e.g., 34-40° F.) water. Generally, it is desirable to inhibit liquid absorption by the absorbent material during the composite forming process. To inhibit liquid absorption, absorbent material can be added to the headbox as an aqueous suspension in chilled water having a temperature in the range from about 0-5° C., preferably from about 0-3° C., and more preferably about 1° C. Alternatively, the absorbent material can be cooled to below 0° C., by placement or storage in a conventional freezer, and then forming a suspension in water, preferably chilled water, immediately prior to composite formation. Limiting the period of time that the absorbent material is in contact with liquid during the forming process also has a positive effect on limiting absorbent material liquid absorption. For embodiments of the composite prepared by this method, the absorbent material suspension is preferably added to the headbox within about 10 seconds, and more preferably within about 5 seconds after preparing the suspension.

By limiting the liquid absorption by the absorbent material during the formation process, composite drying energy and/or time, and the consequent associated expense can be greatly reduced. This advantage can result in composite forming processes that are cost effective and can represent significant savings for consumer absorbent products such as diapers, feminine care products, and adult incontinence products.

Once the headbox contents have been deposited onto the foraminous support, the dispersion medium begins to drain from the deposited slurry to provide an at least partially dewatered fibrous web. Removal of the dispersion medium (e.g., water) from the deposited fibrous slurry or slurries (i.e., the partially dewatered web) continues through, for example, the application of pressure, vacuum, and combinations thereof, and results in the formation of a wet composite.

The composite is ultimately produced by drying the wet composite. Drying removes at least a portion of the remaining dispersion medium and water and provides an absorbent composite having the desired moisture content. Suitable composite drying methods include, for example, the use of drying cans, air floats and through air dryers. Other drying methods and apparatus known in the pulp and paper industry may also be used. Drying temperatures, pressures and times are typical for the equipment and methods used, and are known to those of ordinary skill in the art in the pulp and paper industry.

For foam methods, the fibrous slurry or slurries are aqueous or foam and further include a surfactant. Suitable surfactants include ionic, nonionic, and amphoteric surfactants known in the art.

The deposition of the components of the absorbent composite onto the foraminous support ultimately results in the formation of a wet composite that includes absorbent material that may have absorbed water and, as a result, swollen in size. Water is withdrawn from the wet composite containing the water-swollen absorbent material distributed on the support and the wet composite dried.

In the forming methods, the absorbent material preferably absorbs less than about 20 times its weight in the dispersion medium, more preferably less than about 10 times, and even more preferably less than about 1 time its weight in the dispersion medium. Absorbent materials that absorb liquid only after prolonged contact with liquid, or that absorb liquid only under certain conditions, and do not absorb any significant amount of liquid during the forming process can also be used.

Foam methods are advantageous for forming the composite for several reasons. Generally, foam methods provide fibrous webs that possess both relatively low density and relatively high tensile strength. For composites composed of substantially the same components, foam-formed composites generally have densities greater than airlaid webs and less than wetlaid webs. Similarly, the tensile strength of foam-formed webs is substantially greater than for airlaid webs and approach the strength of wetlaid webs. Also, the use of foam-forming technology allows better control of the orientation and uniform distribution of fibers and the incorporation of a wide range of materials (e.g., long and synthetic fibers that cannot be readily incorporated into wetlaid processes) into the composite.

Absorbent composites formed in accordance with the present invention can be advantageously incorporated into a variety of absorbent articles such as diapers including disposable diapers and training pants; feminine care products including sanitary napkins, and pant liners; adult incontinence products; toweling; surgical and dental sponges; bandages; food tray pads; and the like. Because the composite can be highly absorbent, the composite can be included into an absorbent article as a liquid storage core. In such a construct, the composite can be combined with one or more other composites or layers including, for example, an acquisition and/or a distribution layer. Alternatively, because the composite can rapidly acquire, distribute, and store liquid, the composite can be effectively incorporated into an absorbent article as the sole absorbent component without including other individual layers such as acquisition and/or distribution layers. In one embodiment, the present invention provides an absorbent article, such as a diaper, that includes an absorbent composite having a liquid pervious facing sheet and a liquid impervious backing sheet. Furthermore, because the composite can have the capacity to rapidly acquire and distribute liquid, the composite can serve as a liquid management layer that acquires and transfers a portion of the acquired liquid to an underlying storage core. Thus, in another embodiment, the absorbent composite can be combined with a storage core to provide an absorbent core that is useful in absorbent articles.

In another aspect, the present invention provides absorbent constructs that include the composite described above. The constructs can be advantageously incorporated into absorbent articles such as personal care absorbent products.

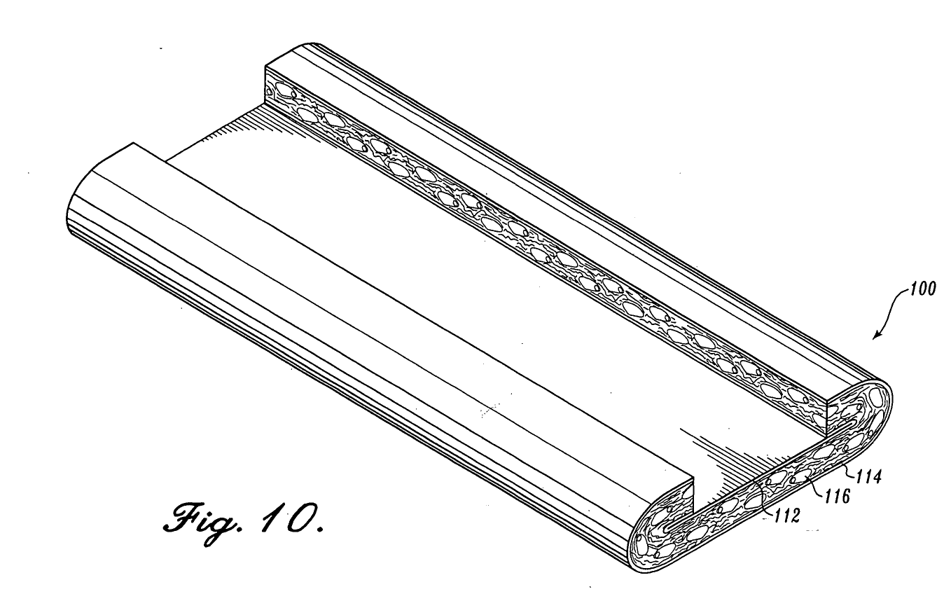

In one embodiment, the construct is a composite as described above that is folded into a C-fold configuration. A perspective view of a representative C-fold composite is illustrated schematically in FIG. 10. Referring to FIG. 10, C-folded composite 100 includes first stratum 112, second stratum 116, and third stratum 114.

The composite can be folded by any one of a variety of methods including those known in the art. As illustrated in FIG. 10, in one embodiment, the C-folded composite has a length greater than about three times its width and is symmetrically folded such that each fold overlays a portion of the unfolded portion of the composite. The composite can be folded such that, on each side of the composite's centerline, about 10 to about 40 percent of the composite's prefolded width remains outside of the folded portion. In one embodiment, the composite has a prefolded width up to about 240 mm and a length up to about 450 mm. In other embodiments, the dimensions of the composite can be-optimized for the particular intended use.

The C-folded composite offers an advantage in liquid acquisition compared to unfolded composites. Liquid is received by the unfolded portion of the composite is wicked away from the initial point of insult. Once the liquid reaches the point at which the composite is overlapped by the fold, the composite presents two surfaces for liquid wicking. Accordingly, the C-folded composite has an increased liquid acquisition rate compared to unfolded composites once the acquired liquid contacts the overlap portion of the C-folded composite.

The composition of the C-folded composite's strata can be widely varied as described above. In one embodiment, the composite has an overall basis weight in the range from about 350 to about 450 gsm and includes first and third strata having basis weights of about 50 gsm and 20 gsm, respectively, and is composed of synthetic fibers (about 80 percent by weight based on the total weight of fibers in the stratum) and bicomponent binder fibers (about 20 percent by weight based on the total weight of fibers in the stratum). The composite's second stratum includes absorbent material present in an amount from about 35 to about 50 percent by weight based on the total weight of the composite and a mixture of matrix and resilient fibers, for example, fluff pulp fibers in an amount from about 40 to about 80 percent by weight, preferably from about 50 to about 70 percent by weight, based on the total weight of fibers in the stratum, and crosslinked cellulosic fibers in an amount from about 20 to about 60 percent by weight, preferably from about 30 to about 50 percent by weight, based on the total weight of fibers in the stratum.

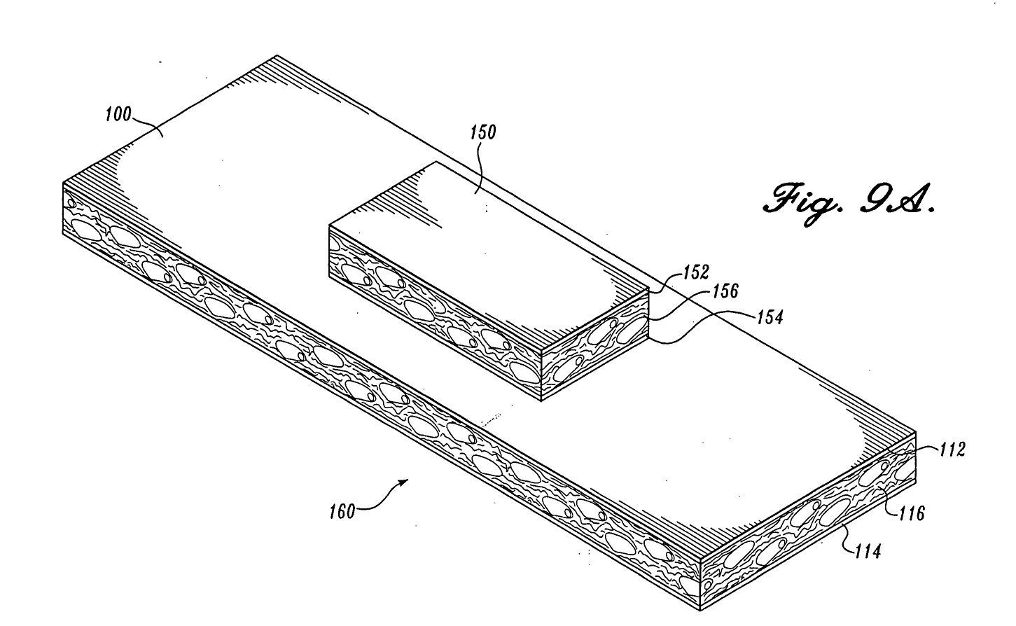

In another embodiment, the invention provides a construct that includes two composites as described above arranged in a pledget/core configuration. In this embodiment, the construct includes a first composite (i.e., pledget) with an adjacent underlying second composite (i.e., core). The lower surface of the first composite is coextensive with at least a portion of the upper surface of the second composite (i.e., the lower surface of the first composite has a surface area less than the upper surface of the second composite). A perspective view of a representative construct having the pledget/core configuration described above is schematically illustrated in FIG. 9A. FIG. 9B is a cross-sectional view of the construct shown in FIG. 9A.

Referring to FIG. 9A, construct 160 includes first composite 150 and adjacent underlying second composite 100. Each of composites 150 and 100 includes first, second, and third strata, 152, 156, and 154, and 112, 116, and 114, respectively. Composite 150 acts as a pledget and is positioned on the construct so as to initially receive liquid from a liquid insult. Composite 150 serves to rapidly acquire and temporarily store liquid, which is then distributed to underlying composite 100. Composite 100 serves as a liquid storage core. Accordingly, the lower surface of first composite 150 (i.e., third stratum 154) and a portion of the upper surface of second composite 100 (i.e., a portion of first stratum 112) are in contact and include components to effectively and efficiently transfer liquid from composite 150 (e.g., second stratum 156) to composite 100 (e.g., second stratum 116).

To effect efficient liquid transfer from the pledget (i.e., composite 150) to the core (i.e., composite 100) strata 154 and 112 include cellulosic fibers, for example, a blend of fluff pulp and crosslinked cellulosic fibers. Construct 160 can be suitably formed from composites described above. Suitable first composites 150 include composites C and D described above, each having relatively low basis weight (e.g., about 20 gsm) third strata composed of a blend of fluff pulp and crosslinked fibers (e.g., about 30 to about 40 percent by weight fluff pulp and about 60 to about 70 percent by weight crosslinked cellulosic fibers). Suitable second composites 100 include composites E and F described above, each having relatively low basis weight (e.g., about 40 gsm) first strata composed of a blend of fluff pulp and crosslinked fibers (e.g., about 40 to about 50 percent by weight fluff pulp and about 50 to about 60 percent by weight crosslinked cellulosic fibers).

To enhance surface dryness, construct 160 includes composite 150 having first stratum 152 that imparts surface dryness and low rewet to the construct. In one such embodiment, stratum 152 serves to rapidly acquire liquid by having a relatively low basis weight (e.g., about 50 gsm) and includes synthetic fibers (e.g., an 80:20 blend of polyethylene terephthalate fibers and bicomponent binder fibers).

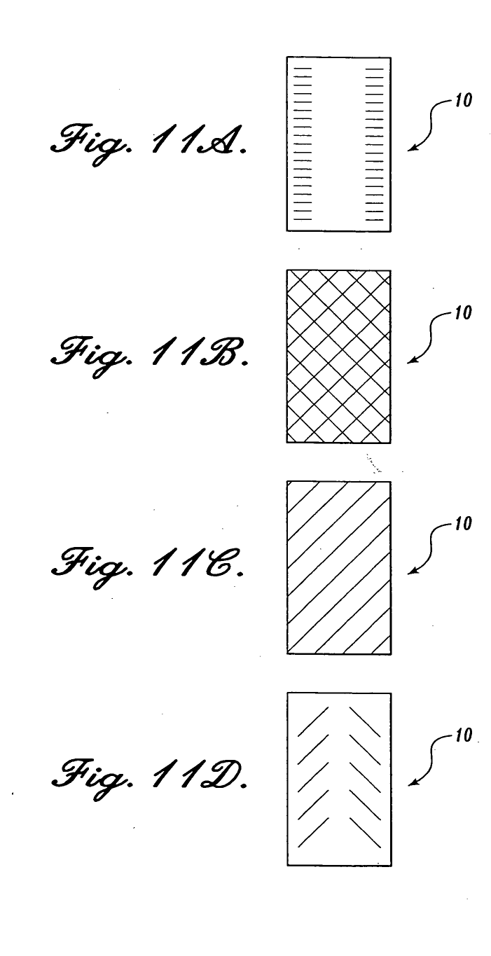

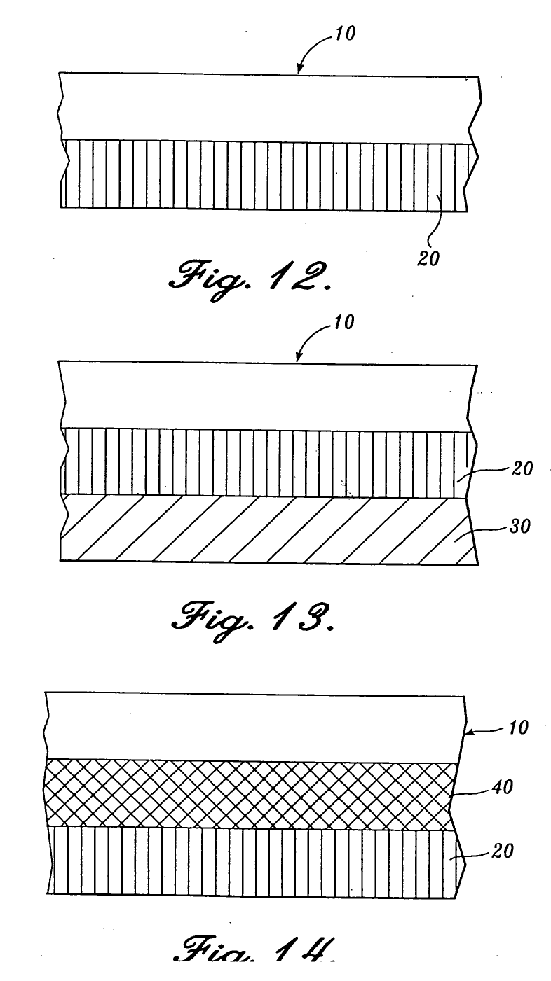

The composite can be incorporated in an absorbent article as the absorbent structure. The absorbent composite can be used alone or, as illustrated in FIG. 12, can be used in combination with one or more other structures. In FIG. 12, the absorbent composite (10) is employed as an upper acquisition/distribution composite in combination with storage structure 20 composed of, for example, a fibrous web that includes superabsorbent material. Storage structure 20, if desired, can also include densified, bonded cellulose fibers. As illustrated in FIG. 13, third structure 30 (e.g., a core or retention structure) can also be employed, if desired, with storage structure 20 and composite 10. If desired, retention structure 30 can also be composed of a fibrous web including superabsorbent material such as, for example, a fibrous web or densified bonded cellulose fibers. Alternatively, a distribution structure 40 can be interposed between composite 10 and storage structure 20 as illustrated in FIG. 14. Distribution structure 40 is generally a hydrophilic fibrous material that includes, for example, hydrophilic fibers such as cellulosic fibers, preferably crosslinked cellulosic fibers, and a binder. In one preferred embodiment, the cellulosic fibers are crosslinked eucalyptus fibers. Distribution structure 40 can optionally include superabsorbent polymeric material.

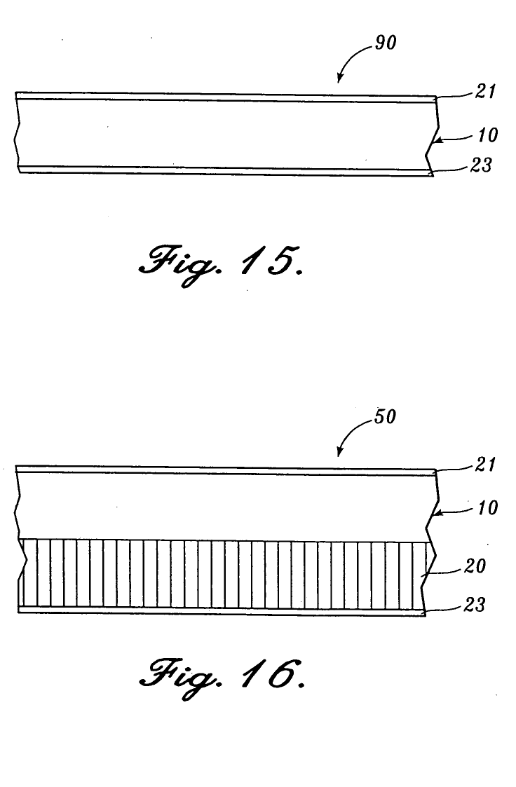

A variety of suitable absorbent articles can be produced from the composite of the invention. The most common include absorptive consumer products such as diapers, feminine hygiene products such as feminine napkins, and adult incontinence products. The composite of the invention can be used alone, or in combination with other structures, layers, or composites, to provide an absorbent structure for incorporating into an absorbent article. For example, referring to FIG. 15, absorbent article 90 includes representative composite 10, topsheet 21, and backsheet 23. In all of the absorbent articles described herein, the composite is generally secured within the topsheet and backsheet, which can be secured to each other. Referring to FIG. 16, absorbent article 50 includes composite 10 and underlying storage structure 20. Liquid pervious facing sheet 21 overlies composite 10 and liquid impervious backing sheet 23 underlies storage structure 20. The composite provides advantageous liquid acquisition performance for use in, for example, diapers. The capillary structure of the composite aids in fluid transport in multiple wettings. Generally, storage structure 20 includes a fibrous web, for example, a strengthened web of cellulose fibers, and may also incorporate additives, such as superabsorbent polymers to significantly increase the absorbent capacity of storage structure 20.

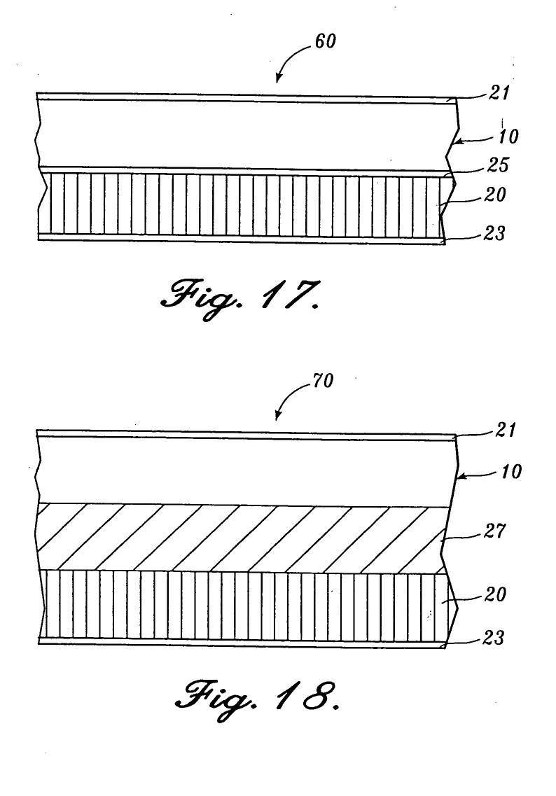

The article in FIG. 16 is shown for purposes of exemplifying a typical absorbent article, such as a diaper or feminine napkin. One of ordinary skill will be able to make a variety of different absorbent constructs using the concepts taught herein. For example, a typical construction for an adult incontinence absorbent structure is shown in FIG. 17. Article 60 includes facing sheet 21, absorbent composite 10, storage structure 20, and backing sheet 23. Facing sheet 21 is pervious to liquid while backing sheet 23 is impervious to liquid. In this construct, liquid pervious tissue 25 composed of a polar, fibrous material is positioned between absorbent composite 10 and storage structure 20.

Referring to FIG. 18, another absorbent article 70 includes backing sheet 23, storage structure 20, intermediate structure 27, absorbent composite 10, and facing sheet 21. Intermediate structure 27 contains, for example, a densified fibrous material such as a combination of cellulose acetate and triacetin, which are combined just prior to forming the article. Intermediate structure 27 can thus bond to both absorbent composite 10 and storage structure 20 to form an absorbent article with much more integrity than one in which the absorbent composite and storage structure are not bonded to each other. The hydrophilicity of structure 27 can be adjusted in such a way as to create a hydrophilicity gradient among structures 10, 27, and 20. It should be understood that an independent intermediate structure is not required in order to get structure-to-structure bonding. When one of two adjacent structures or both structures contain a binder, if the two structures are brought together when the bonding medium is still active, bonding between the two structures will occur and provide a stronger composite compared to a composite lacking any bonding. Alternatively, intermediate structure 27 can be a distribution structure as described above in reference to the construct of FIG. 14.

The composite of the present invention improves the surface dryness rewet performance, acquisition rate, and softness, of absorbent products and articles that incorporate the absorbent composite. The absorbent composite also provides increased pad integrity, improved appearance, and a reduction in wet collapse during use for absorbent products that incorporate the absorbent composite. The composite also offers the advantage of enhanced retention of superabsorbent particulate material. Furthermore, because the composite can be manufactured and delivered in web form, absorbent product manufacturing processes that include the absorbent composite are simplified relative to manufacturing processes that involve the handling of bales of crosslinked fibers or fluff pulp. Thus, in addition to the increased performance provided to absorbent products that incorporate the absorbent composite of this invention, the absorbent composite offers economic advantages over the combination of separate layers of high-loft nonwoven fibers and crosslinked cellulosic fibers.

While the preferred embodiment of the invention has been illustrated and described, it will be appreciated that various changes can be made therein without departing from the spirit and scope of the invention.

1-107. (canceled)

108. An absorbent composite comprising a first stratum, a second stratum, a third stratum, a first transition zone intermediate and coextensive with the first and second strata, and a second transition zone intermediate and coextensive with the second and third strata;
the first stratum comprising at least one of synthetic fibers, matrix fibers, and resilient fibers
the second stratum comprising absorbent material and at least one of matrix fibers and resilient fibers;
the third stratum comprising at least one of synthetic fibers, matrix fibers, and resilient fibers;
the first transition zone comprising fibers from the first and second strata commingled substantially uniformly across the composite's width and along the composite's length; and
the second transition zone comprising fibers from the second and third strata commingled substantially uniformly across the composite's width and along the composite's length.

109. The composite of claim 1, wherein the resilient fibers comprise fibers selected from the group consisting of chemically stiffened fibers, anfractuous fibers, chemithermomechanical pulp fibers, prehydrolyzed kraft pulp fibers, synthetic fibers, crosslinked cellulosic fibers, and mixtures thereof.

110. The composite of claim 1, wherein the synthetic fibers comprise fibers selected from the group consisting of polyolefin, polyester, polyamide, polyethylene terephthalate and thermobondable fibers.

111. The composite of claim 1, wherein the matrix fibers comprise fibers selected from the group consisting of cellulosic fibers, wood pulp fibers, cotton linters, cotton fibers, hemp fibers, rayon fibers, cellulose acetate fibers, and mixtures thereof.

112. The composite of claim 1, wherein one or more strata further comprises a binder selected from the group consisting of thermoplastic fibers, soluble bonding mediums, bicomponent binding fibers, wet strength agents, and a poly-amide-epichlorohydrin resin.

113. The composite of claim 1, wherein the absorbent material comprises a superabsorbent polymer.

114. The composite of claim 4, wherein the wood pulp fibers are present in the first stratum in an amount from about 20 to about 80 percent by weight based on the total weight of fibers in the stratum.

115. The composite of claim 2, wherein the crosslinked cellulosic fibers are present in the first stratum in an amount from about 20 to about 80 percent by weight based on the total weight of fibers in the stratum.

116. The composite of claim 1, wherein the second stratum comprises from about 20 to about 80 percent by weight absorbent material based on the total weight of the stratum.

117. The composite of claim 3, wherein the polyethylene terephthalate fibers are present in the third stratum in an amount from about 70 to about 90 percent by weight based on the total weight of fibers in the stratum.

118. The composite of claim 5, wherein the bicomponent binding fibers are present in the third stratum in an amount from about 10 to about 30 percent by weight based on the total weight of fibers in the stratum.

119. The composite of claim 4, wherein the wood pulp fibers are present in any one of the second or the third stratum in an amount from about 30 to about 80 percent by weight based on the total weight of fibers in the stratum.

120. The composite of claim 2, wherein the crosslinked cellulosic fibers are present in any one of the second or the third stratum in an amount from about 20 to about 70 percent by weight based on the total weight of fibers in the stratum.

121. A method for forming a fibrous web, comprising the steps of:
(a) forming a first fibrous furnish comprising fibers in an aqueous dispersion medium;
(b) forming a second fibrous furnish comprising fibers in an aqueous dispersion medium;
(c) moving a first foraminous element in a first path;
(d) moving a second foraminous element in a second path;
(e) passing the first fibrous furnish into contact with the first foraminous element moving in the first path;
(f) passing the second fibrous furnish into contact with the second foraminous element moving in the second path;
(g) passing a third fibrous furnish between the first and second furnishes; and
(h) withdrawing liquid from the first, second, and third fibrous furnishes through the first and second foraminous elements to provide a fibrous web.

122. The method of claim 14, wherein the first fibrous furnish comprises synthetic fibers and bicomponent binder fibers.

123. The method of claim 14, wherein the second fibrous furnish comprises matrix fibers, resilient fibers, absorbent material and a binder.

124. The method of claim 14, wherein the third fibrous furnish comprises synthetic fibers and binder fibers.

125. The method of claim 14, wherein the third fibrous furnish comprises matrix fibers and resilient fibers.
